(19) United States
(12) Patent Application Publication
(10) Pub. No.: US 2002/0193564 A1
(43) Pub. Date: Dec. 19, 2002

(54) Oligomeric chaperone proteins

(76) Inventors: Fergal Conan Hill, Les Martres de Veyre (FR); Jean Chatellier, Les Martres de Veyre (FR); Alan Roy Fersht, Cambridge (GB)

Correspondence Address: FROMMER LAWRENCE & HAUG, 745 FIFTH AVENUE- 10TH FL., NEW YORK, NY, 10151, US

(21) Appl. No.: 10/007,314

(22) Filed: Nov. 8, 2001

Related U.S. Application Data
(63) Continuation-in-part of application No. PCT/GB00/01822, filed on May 12, 2000.

(30) Foreign Application Priority Data
May 14, 1999 (GB) 9911298.9
Dec. 23, 1999 (GB) 9930530.2

(51) Int. Cl. C07K 14/005; C12N 7/00

(52) U.S. Cl. 530/350; 435/235.1

(57) ABSTRACT

The invention relates to a polypeptide monomer capable of oligomerisation, said monomer comprising a polypeptide which potentiates protein folding inserted into the sequence of a subunit of an oligomerisable protein scaffold.

This application is a Continuation-in-Part of PCT/GB00/01822, filed May 12, 2000, designating the U.S., published Nov. 23, 2000 as WO 00/69886 and claiming priority from GB 9911298.9 filed May 14, 1999 and GB 9930530.2 filed Dec. 23, 1999. All of the above-mentioned applications, as well as all documents cited herein and documents referenced or cited in documents cited herein, are hereby incorporated herein by reference.

The present invention relates to oligomers of chaperone or minichaperone proteins. In particular, the invention relates to polypeptides constructed by oligomerisation of minichaperone monomers to form a heptameric ring structure.

Proteins, especially catalytic proteins (enzymes) and proteins which have biological activities, are dependent on tertiary structure for most or all of their functional attributes. Tertiary structure, which is defined by the three-dimensional arrangement of the protein, is dependent on the folding of the primary polypeptide sequence in three dimensions. The tertiary structure is stabilised by the interaction between parts of the primary sequence in the folded state, such as the formation of disulphide bonds, and energetic considerations deriving from juxtaposition of particular chemical entities in the three-dimensional arrangement.

It is known that the tertiary structure of proteins, particularly proteins which are stored for any length of time, can degrade, leading to suboptimal activity. This can be due to a variety of factors, including aggregation and the formation of improper intra- and inter-molecular interactions. Moreover, it is also known that proteins produced by recombinant DNA technology are frequently misfolded, especially if produced in bacterial expression systems. The strongly reducing environment present in bacterial cytoplasm impedes the formation of correct disulphide bonds, thus obstructing the folding process.

Many proteins require the assistance of molecular chaperones in order to be fold in vivo or to be refolded in vitro in high yields. Molecular chaperones are proteins, which are often large and require an energy source such as ATP to function. A key molecular chaperone in Escherichia coli is GroEL, which consists of 14 subunits each of some 57.5 kD molecular mass arranged in two seven membered rings. There is a large cavity in the GroEL ring system, and it is widely believed that the cavity is required for successful protein folding activity. For optimal activity, a co-chaperone, GroES, is required which consists of a seven membered ring of 10 kD subunits. The activity of the GroEL/GroES complex requires energy source ATP.

Some proteins are monomers, consisting of a single subunit. Many proteins are oligomeric, consisting of more than one subunit. Sometimes the subunits are identical, sometimes there are different types of subunits. Frequently, the subunits are linked non-covalently. Sometimes, the subunits are attached covalently, with a stretch of polypeptide linking the C-terminus of one domain to the N-terminus of another.

Allosteric proteins are a special class of oligomeric proteins, which alternate between two or more different three-dimensional structures on the binding of ligands and substrates. Allosteric proteins are often involved in control processes in biology or where mechanical and physico-chemical energies are interconverted.

GroEL is an allosteric protein. The role of ATP is to trigger this allosteric change, causing GroEL to convert from a state that binds denatured proteins tightly to one that binds denatured proteins weakly. The co-chaperone, GroES, aids in this process by favouring the weak-binding state. It may also act as a cap, sealing off the cavity of GroEL. Further, its binding to GroEL is likely directly to compete with the binding of denatured substrates. The net result is that the binding of GroES and ATP to GroEL which has a substrate bound in its denatured form is to release the denatured substrate either into the cavity or into solution where it can refold.

Minichaperones have been described in detail elsewhere (see International patent application WO99/05163, the disclosure of which in incorporated herein by reference). Minichaperone polypeptides possess chaperoning activity when in monomeric form and do not require energy in the form of ATP. Defined fragments of the apical domain of GroEL of approximately 143-186 amino acid residues in length have molecular chaperone activity towards proteins either in solution under monomeric conditions or when monodisperse and attached to a support.

The activity of minichaperones, although sufficient for many purposes, is inferior to that of intact GroEL. There is thus a need for a more active form of minichaperone, which nevertheless retains independence from energy requirements.

### SUMMARY OF THE INVENTION

The activity of minichaperones, although sufficient for many purposes, is inferior to that of intact GroEL. It is postulated that this could be due to the inability of GroES to oligomerise. There is thus a widespread requirement for a system which would allow the oligomerisation of polypeptides to form functional protein oligomers which have activities which surpass those of recombinant monomeric polypeptides.

According to the present invention, there is provided a polypeptide monomer capable of oligomerisation. said monomer comprising a polypeptide sequence which potentiates protein folding inserted into the sequence of a subunit of an oligomerisable protein scaffold.

It has been observed that the oligomerisation of polypeptide sequences which potentiate protein folding polypeptides allows their spatial juxtaposition, which potentiates their activity.

A “polypeptide sequence which potentiates protein folding” may any one of a number of polypeptides which are capable of facilitating the correct folding or refolding of proteins or polypeptides in vivo and/or in vitro. For example, the polypeptide sequence which potentiates protein folding may be a minichaperone polypeptide or a foldase. Advantageously, the foldase is selected from the group consisting of thiol/disulphide oxidoreductases and peptidyl prolyl isomerases. Preferably, the thiol/disulphide oxidoreductase is selected from the group consisting of E. coli DsbA and mammalian PDI, or a derivative thereof. Preferably, the peptidyl prolyl isomerase is a cyclophilin.

In a preferred aspect, the polypeptide sequence which potentiates protein folding may be a protease prosequence. Whereas molecular chaperones do not provide steric information necessary for newly synthesised proteins to fold correctly, but minimise the risk that partially folded protein chains will aggregate with one another, protease prosequences, which are intramolecular chaperones, provide steric information indispensable for the rest of the protein to fold correctly.

A “minichaperone polypeptide”, as referred to herein, refers to a minichaperone polypeptide as described in WO99/05163, the disclosure of which in incorporated herein by reference. The minichaperone polypeptide preferably comprises fragments of a molecular chaperone, preferably fragments of any hsp-60 chaperone, and may be selected from the group consisting of mammalian hsp-60 and GroEL, or a derivative thereof.

In the case that the fragment is a fragment of GroEL, it advantageously does not have an Alanine residue at position 262 and/or an Isoleucine residue at position 267 of the sequence of intact GroEL as defined at GenBank Accession No. P06159. Preferably, it has a Leucine residue at position 262 and/or a Methionine residue at position 267 of the sequence of intact GroEL. The invention therefore encompasses the use of a fragment of GroEL comprising a Leucine residue at position 262 and/or a Methionine residue at position 267 of the sequence of intact GroEL for potentiating the folding of a polypeptide.

In a preferred embodiment, the minichaperone comprises a region which is homologous to at least one of fragments 191-376, 191-345 and 191-335 of the sequence of intact GroEL.

A protein scaffold is a protein, or part thereof, whose function is to determine the structure of the protein itself, or of a group of associated proteins or other molecules. Scaffolds therefore have a defined three-dimensional structure when assembled, and have the capacity to support molecules or polypeptide domains in or on the said structure. Advantageously, a scaffold has the ability to assume a variety of viable geometries, in relation to the three-dimensional structure of the scaffold and/or the insertion site of the polypeptides.

Preferably, the scaffold according to the invention is a chaperonin cpn10/Hsp10 scaffold. Cpn10 is a widespread component of the cpn60/cpn10 chaperonin system. Examples of cpn10 include bacterial GroES and bacteriophage T4 Gp31. Further members of the cpn10 family will be known to those skilled in the art.

The invention moreover comprises the use of derivatives of naturally-occurring scaffolds. Derivatives of scaffolds (including scaffolds of the cpn10 and 60 families) comprise mutants thereof, which may contain amino acid deletions, additions or substitutions (especially replacement of Cys residues in Gp31), hybrids formed by fusion of different members of the Cpn10 or 60 families and/or circular permutated protein scaffolds, subject to the maintenance of the “oligomerisation” property described herein.

Protein scaffold subunits assemble to form a protein scaffold. In the context of the present invention, the scaffold may have any shape and may comprise any number of subunits. Preferably, the scaffold comprises between 2 and 20 subunits, advantageously between 5 and 15 subunits, and ideally about 10 subunits. The scaffold of cpn10 family members comprises seven subunits, in the shape of a seven-membered ring or annulus. Advantageously, therefore, the scaffold is a seven-membered ring.

Advantageously, the polypeptide sequence which potentiates protein folding is inserted into the sequence of the oligomerisable protein scaffold subunit such that both the N and C termini of the polypeptide monomer are formed by the sequence of the oligomerisable protein scaffold subunit. Thus, the polypeptide is included with the sequence of the scaffold subunit, for example by replacing one or more amino acids thereof.

It is known that cpn10 subunits possess a “mobile loop” within their structure. The mobile loop is positioned between amino acids 15 and 34, preferably between amino acids 16 to 33, of the sequence of E. coli GroES, and equivalent positions on other members of the cpn10 family. The mobile loop of T4 Gp31 is located between residues 22 to 45, advantageously 23 to 44. Advantageously, the polypeptide sequence which potentiates protein folding is inserted by replacing all or part of the mobile loop of a cpn10 family polypeptide.

Where the protein scaffold subunit is a cpn10 family polypeptide, the polypeptide sequence which potentiates protein folding may moreover be incorporated at the N or C terminus thereof, or in positions which are equivalent to the roof β hairpin of cpn10 family peptides. This position is located between positions 54 and 67, advantageously 55 to 66, and preferably 59 and 61 of bacteriophage T4 Gp31, or between positions 43 to 63, preferably 44 to 62, advantageously 56 to 57 of E. coli GroES.

Advantageously, the polypeptide sequence which potentiates protein folding may be inserted at an N or C terminus of a scaffold subunit in association with circular permutation of the subunit itself. Circular permutation is described in Graf and Schachmran, PNAS(USA) 1996, 93:11591. Essentially, the polypeptide is circularised by fusion of the existing N and C termini, and cleavage of the polypeptide chain elsewhere to create novel N and C termini. In a preferred embodiment of the invention, the polypeptide may be included at the N and/or C terminus formed after circular permutation. The site of formation of the novel termini may be selected according to the features desired, and may include the mobile loop and/or the roof β hairpin.

Advantageously, polypeptide sequences which potentiates protein folding, which may be the same or different, may be inserted at more than one of the positions above-identified in the protein scaffold subunit. Thus, each subunit may comprise two or more polypeptides, which are displayed on the scaffold when this is assembled.

Polypeptide sequences which potentiate protein folding may be displayed on a scaffold subunit in free or constrained form, depending on the degree of freedom provided by the site of insertion into the scaffold sequence. For example, varying the length of the sequences flanking the mobile loop in the scaffold will modulate the degree of constraint of any polypeptide inserted therein.

In a second aspect, the invention relates to a polypeptide oligomer comprising two or more monomers according to the first aspect of the invention. The oligomer may be configured as a heterooligomer, comprising two or more different polypeptide sequences which potentiate protein folding inserted into the scaffold, or as a homooligomer, in which the polypeptides inserted into the scaffold are the same.

If the oligomer according to the invention is a heterooligomer, it may be configured such that the polypeptides juxtaposed thereon have complementary biological activities. For example, one or more minichaperones and foldases are advantageously displayed on the same scaffold, enabling them to act in concert.

The monomers which constitute the oligomer may be covalently crosslinked to each other. Cross linking may be performed by recombinant approaches, such that the monomers are expressed ab initio as an oligomer; alternatively, cross-linking may be performed at Cys residues in the scaffold. For example, unique Cys residues inserted between positions 56 and 57 of the GroES scaffold, or equivalent positions on other members of the cpn10 family, may be used to cross-link scaffold subunits.

In a third aspect, the present invention relates to a method for preparing a polypeptide monomer capable of oligomerisation according to the first aspect of the invention, comprising the steps of inserting a nucleic acid sequence encoding a polypeptide sequence which potentiates protein folding into a nucleic acid sequence encoding a subunit of an oligomerisable protein scaffold, incorporating the resulting nucleic acid into an expression vector, and expressing the nucleic acid to produce the polypeptide monomers.

Oligomerised protease prosequences form polyvalent steric chaperones. The invention therefore provides polyvalent prosequence polypeptides, optionally incorporating one or more minichaperones and/or foldases, which are useful in refolding polypeptides in vitro and in vivo. Oligomerised prosequences may be used to potentiate the folding of, inter alia, proteases. Moreover, polyvalent prosequence polypeptides may be used to alter the folding patterns of polypeptides, thus permanently altering their activities.

The invention moreover relates to a method for producing a polypeptide oligomer according to the second aspect of the invention, comprising allowing the polypeptide monomers produced as above to associate into an oligomer. Preferably, the monomers are cross-linked to form the oligomer.

According to a fourth aspect of the present invention, there is provided a method for potentiating the folding of a polypeptide comprising contacting the polypeptide with a multimeric minichaperone polypeptide as described above.

The polypeptide which is folded by the method of the invention is preferably an unfolded or misfolded polypeptide, and advantageously comprises a disulphide.

The invention moreover concerns a method as described above wherein the oligomer is immobilised onto a solid phase support, which may be agarose. Accordingly, the invention also provides a solid phase support having immobilised thereon an oligomer according to the present invention, and a column packed at least in part with such a solid phase support.

The oligomer according to the invention may be combined with different oligomers, or with independent molecules capable of potentiating protein folding. For example, the oligomers according to the invention may be used, in solution or in immobilised form, in combination with foldases, chaperones or other enzymes.

### BRIEF DESCRIPTION OF THE DRAWINGS

FIG. 1. (a) Three-dimensional structure of Gp31 of bacteriophage T4 solved at 2.3 Å. Positions mentioned in the text are indicated (residues numbered as in van der Vies, S., Gatenby, A. & Georgopoulos, C. (1994) Nature 368, 654-656). (b) Three-dimensional structure of minichaperone GroEL(191-376) solved at 1.7 Å. The distance between residues 25 and 43 of Gp31 is around 12 Å; the distance between residues 191 and 376 of GroEL is around 9 Å. Positions mentioned in the text are indicated (residues numbered as in Hemmingsen, S. M., Woolford, C., van, d. V. S., Tilly, K., Dennis, D. T., Georgopoulos, C. P., Hendrix, R. W. & Ellis, R. J. (1988) Nature 333, 330-334.). Secondary structure representations are drawn with MolScript (Kraulis, P. (1991) J. Appl. Crystallogr. 24, 946-950).

FIG. 2. Schematic representation of Gp31 proteins in the vectors used in this study. The presence of the Gp3 I mobile loop (residues 23 to 44) and/or minichaperone GroEL (residues 191 to 376) are indicated by boxes. The nucleotide sequence of the Gp31 mobile loop and relevant restriction sites are shown. The names of the corresponding vector are listed in the left margin.

FIG. 3. (a) Molecular weight determination by analytical gel filtration chromatography. Wild-type proteins Gp31 (Mᵣ≈7×12 kDa) and GroEL(191-376) (Mᵣ≈22 kDa) and, Gp31Δloop and Gp31Δ::GroEL(191-376) (MC₇) were run on a Superdex™ 200 (HR 10/30) column (Pharmacia Biotech.) calibrated with molecular weight standards (solid-line and circles). Gp31Δloop and MC₇ eluted at volumes corresponding to molecular weights of ≈145.6 and ≈215 kDa, respectively. (b) Molecular weight determination of MC₇ by equilibrium analytical ultracentrifugation. The apparent molecular weight of MC₇ is ≈215 kDa.

FIG. 4. Characterisation of MC₇ by CD spectroscopy. (a) Far UV-CD spectrum at 25° C. (b) Thermal denaturation followed at 222 nm at a heating rate of 1° C.min⁻¹.

FIG. 5. (a) Binding specificity of MC₇ to GroES determined by ELISA. (b) Inhibition of MC₇ binding to heptameric co-chaperonin GroES by varying concentrations of synthetic peptide corresponding to residues 16 to 32 of GroES mobile loop determined by competition ELISA.

FIG. 6. Binding avidity of MC₇ to anti-GroEL antibodies determined (a) by direct ELISA or (b) by indirect ELISA.

FIG. 7. In vitro refolding of heat- and dithiothreitol-denatured mtMDH. (a) Protection of aggregation at 47° C. followed by light scattering at 550 nm. (b) Time-dependent reactivation of mtMDH at 25° C. (c) Yields of mtMDH reactivation.

FIG. 8 and FIG. 9 show the possible insertion sites for polypeptides in bacteriophage T4 Gp31.

FIG. 10 illustrates the potential attachment sites for polypeptides to a circular scaffold, in this case Gp31.

### DETAILED DESCRIPTION OF THE INVENTION

Definitions

Oligomerisable Scaffold.

An oligomerisable scaffold, as referred to herein, is a polypeptide which is capable of oligomerising to form a scaffold and to which a polypeptide may be fused, preferably covalently, without abolishing the oligomerisation capabilities. Thus, it provides a “scaffold” using which polypeptides may be arranged into multimers in accordance with the present invention. Optionally, parts of the wild-type polypeptide from which the scaffold is derived may be removed, for example by replacement with the polypeptide which is to be presented on the scaffold.

Monomer.

Monomers according to the present invention are polypeptides which possess the potential to oligomerise. This is brought about by the incorporation, in the polypeptide, of an oligomerisable scaffold subunit which will oligomerise with further scaffold subunits if combined therewith.

Oligomer.

As used herein, “oligomer” is synonymous with “polymer” or “multimer” and is used to indicate that the object in question is not monomeric. Thus, oligomeric polypeptides according to the invention comprise at least two monomeric units joined together covalently or non-covalently. The number of monomeric units employed will depend on the intended use of the oligomer, and may be between 2 and 20 or more. Advantageously, it is between 5 and 10, and preferably about 7.

Polypeptide.

As used herein, a polypeptide is a molecule comprising at lest one peptide bond linking two amino acids. This term is synonymous with “protein” and “peptide”, both of which are used in the art to describe such molecules. A polypeptide may comprise other, non-amino acid components. The polypeptide the folding of which is potentiated by the method of the invention may be any polypeptide. Preferably, however, it is an unfolded or misfolded polypeptide which is in need of folding. Alternatively, however, it may be a folded polypeptide which is to be maintained in a folded state (see below).

Preferably, the polypeptide which is folded according to the invention contains at least one disulphide. Such polypeptides may be referred to herein as disulphide-containing polypeptides.

Examples of polypeptides include those used for medical or biotechnological use, such as interleukins, interferons, antibodies and their fragments, insulin, transforming growth factor, and many toxins and proteases.

Potentiating the Folding.

The invention envisages at least two situations. A first situation is one in which the polypeptide to be folded is in an unfolded or misfolded state, or both. in this case, its correct folding is potentiated by the method of the invention. A second situation is one in which the polypeptide is substantially already in its correctly folded state, that is all or most of it is folded correctly or nearly correctly. In this case, the method of the invention serves to maintain the folded state of the polypeptide by affecting the folded/unfolded equilibrium so as to favour the folded state. This prevents loss of activity of an already substantially correctly folded polypeptide. These, and other, eventualities are covered by the reference to “potentiating” the folding of the polypeptide.

Contacting.

The reagents used in the method of the invention require physical contact with the polypeptides whose folding is to be potentiated. This contact may occur in free solution, in vitro or in vivo, with one or more components of the reaction immobilised on solid supports. In a preferred aspect, the contact occurs with the minichaperone oligomer and/or the thiol/disulphide oxidoreductase immobilised on a solid support, for example on a column. Alternatively, the solid support may be in the form of beads or another matrix which may be added to a solution comprising a polypeptide whose folding is to be potentiated.

Fragment.

When applied to chaperone molecules, a fragment is anything other that the entire native molecular chaperone molecule which nevertheless retains chaperonin activity. Advantageously, a fragment of a chaperonin molecule remains monomeric in solution. Preferred fragments are described below. Advantageously, chaperone fragments are between 50 and 200 amino acids in length, preferably between 100 and 200 amino acids in length and most preferably about 150 amino acids in length. Fragments of chaperone molecules which remain monomeric in solution and possess a chaperoning activity which is not energy-dependent are referred to as minichaperones.

Unfolded.

As used herein, a polypeptide may be unfolded when at least part of it has not yet acquired is correct or desired secondary or tertiary structure. A polypeptide is misfolded when it has acquired an at least partially incorrect or undesired secondary or tertiary structure.

Immobilised, Immobilising.

Permanently attached, covalently or otherwise. In a preferred aspect of the present invention, the term “immobilise”, and grammatical variations thereof, refer to the attachment of molecular chaperones or, preferably, foldase polypeptides to a solid phase support using a method as described in WO99/05163.

Solid (Phase) Support.

Reagents used in the invention may be immobilised onto solid phase supports. This means that they are permanently attached to an entity which remains in a different (solid) phase from reagents which are in solution. For example, the solid phase could be in the form of beads, a “polypeptide chip”, a resin, a matrix, a gel, the material forming the walls of a vessel or the like. Matrices, and in particular gels, such as agarose gels, may conveniently be packed into columns. A particular advantage of solid phase immobilisation is that the reagents may be removed from contact with the polypeptide(s) with facility.

Foldase.

In general terms, a foldase is an enzyme which participates in the potentiation of protein folding through its enzymatic activity to catalyse the rearrangement or isomerisation of bonds in the folding polypeptide. They are thus distinct from a molecular chaperone, which bind to polypeptides in unstable or non-native structural states and potentiate correct folding without enzymatic catalysis of bond rearrangement. Many classes of foldase are known, and they are common to animals, plants and bacteria. They include peptidyl prolyl isomerases and thiol/disulphide oxidoreductases. The invention comprises the use of all foldases which are capable of potentiating protein folding through covalent bond rearrangement.

Moreover, as used herein, the term “a foldase” includes one or more foldases. In general, in the present specification the use of the singular does not preclude the presence of a plurality of the entities referred to, unless the context specifically requires otherwise.

Thiol/disulphide Oxidoreductase.

As the name implies, thiol/disulphide oxidoreductases catalyse the formation of disulphide bonds and can thus dictate the folding rate of disulphide-containing polypeptides. The invention accordingly comprises the use of any polypeptide possessing such an activity. This includes chaperone polypeptides, or fragments thereof, which may possess PDI activity (Wang & Tsou, (1998) FEBS lett. 425:382-384). In Eukaryotes, thiol/disulphide oxidoreductases are generally referred to as PDIs (protein disulphide isomerases). PDI interacts directly with newly synthesised secretory proteins and is required for the folding of nascent polypeptides in the endoplasmic reticulum (ER) of eukaryotic cells. Enzymes found in the ER with PDI activity include mammalian PDI (Edman et al., 1985, Nature 317:267, yeast PDI (Mizunaga et al. 1990, J. Biochem. 108:848), mammalian ERp59 (Mazzarella et al., 1990, J. Biochem. 265:1094), mammalian prolyl-4-hydroxylase (Pihlajaniemi et al., 1987, EMBO J. 6: 643) yeast GSBP (Lamantia et al., 1991, Proc. Natl. Acad. Sci. USA, 88:4453) and mammalian T3BP (Yamauchi et al., 1987, Biochem. Biophys. Res. Commun. 146:1485), A. niger PdiA (Ngiam et al., (1997) Curr. genet. 31:133-138) and yeast EUGI (Tachibana et al., 1992, Mol. Cell Biol. 12, 4601). In prokaryotes, equivalent proteins exist, such as the DsbA protein of E. coli. Other peptides with similar activity include, for example, p52 from T. cruzi (Moutiez et al, (1997) Biochem. J. 322:43-48). These polypeptides, and other functionally equivalent polypeptides, are included with the scope of the present invention, as are derivatives of the polypeptides which share the relevant activity (see below). Preferably, the thiol/disulphide oxidoreductase according to the invention is selected from the group consisting of mammalian PDI or E. coli DsbA.

Peptidyl-prolyl Isomerase.

Peptidyl-prolyl isomerases are known enzymes widely present in a variety of cells. Examples include cyclophilin (see, for example, Bergsma et al. (1991) J. Biol. Chem. 266:23204-23214), parbulen, SurA (Rouviere and Gross, (1996) Genes Dev. 10:3170-3182) and FK506 binding proteins FKBP51 and FKBP52. PPI is responsible for the cis-trans isomerisation of peptidyl-prolyl bonds in polypeptides, thus potentiating correct folding. The invention includes any polypeptide having PPI activity. This includes chaperone polypeptides, or fragments thereof, which may possess PPI activity (Wang & Tsou, (1998) FEBS lett. 425:382-384).

Molecular Chaperone.

Chaperones, or chaperoning, are polypeptides which potentiate protein folding by non-enzymatic means, in that they do not catalyse the chemical modification of any structures in folding polypeptides, by potentiate the correct folding of polypeptides by facilitating correct structural alignment thereof. Molecular chaperones are well known in the art, several families thereof being characterised. The invention is applicable to any molecular chaperone molecule, which term includes, for example, the molecular chaperones selected from the following non-exhaustive group:

| | |
|---|---|
| p90 Calnexin | Salopek et al., J. Investig Dermatol Symp Proc (1996) 1:195 |
| HSP family | Walsh et al., Cell Mol. Life Sci. (1997) 53:198 |
| HSP 70 family | Rokutan et al, J. Med. Invest. (1998) 44:137 |
| DNA K | Rudiger et al., Nat. Struct. Biol. (1997) 4:342 |
| DNAJ | Cheetham et al., Cell Stress Chaperones (1998) 3:28 |
| HSP 60 family; GroEL | Richardson et al., Trends Biochem. (1998) 23:138 |
| ER-associated chaperones | Kim et al., Endocr Rev (1998) 19:173 |
| HSP 90 | Smith, Biol. Chem. (1988) 379:283 |
| Hsc 70 | Hohfeld, Biol. Chem. (1988) 379:269 |
| sHsps; SecA; SecB | Beissinger et al., Biol. Chem. (1988) 379:245 |
| Trigger factor | Wang et al., FEBS Lett. (1998) 425:382 |
| zebrafish hsp 47, 70 and | Krone et al., Biochem. Cell Biol. (1997) 75:487 |
| 90 | |
| HSP 47 | Nagata, Matrix Biol. (1998) 16:379 |
| GRP 94 | Nicchitta et al., Curr. Opin. Immunol. (1998) 10:103 |
| Cpn 10 | Cavanagh, Rev. Reprod. (1996) 1:28 |
| BiP | Sommer et al., FASEB J. (1997) 11:1227 |
| GRP 78 | Brostrom et al., Prog. Nucl. Acid. res. Mol. Biol. (1998) |
| | 58:79 |
| C1p, FtsH | Suzuki et al., Trends Biochem. Sci. (1997) 22:118 |
| Ig invariant chain | Weenink et al. Immunol. Cell biol. (1997) 75:69 |
| mitochondrial hsp 70 | Horst et al., BBA (1997) 1318:71 |
| EBP | Hinek, Arch. Immunol. Ther. Exp. (1997) 45:15 |
| mitochondrial m-AAA | Langer et al., Experientia (1996) 52:1069 |
| Yeast Ydj 1 | Lyman et al., Experientia (1996) 52:1042 |
| Hsp 104 | Tuite et al., Trends Genet. (1996) 12:467 |
| ApoE | Blain et al., Presse Med. (1996) 25:763 |
| Syc | Wattiau et al, Mol. Microbiol. (1996) 20:255 |
| Hip | Ziegelhoffer et al., Curr. Biol. (1996) 6:272 |
| TriC family | Hendrick et al., FASEB J. (1995) 9:1559 |
| CCT | Kubota et al., Eur. J. Biochem. (1995) 230:3 |
| PapD, calmodulin | Stanfield et al., Curr. Opin. Struct. Biol. (1995) 5:103 |

Two major families of protein folding chaperones which have been identified, the heat shock protein 60 (hsp60) class and the heat shock protein 70 (hsp70) class, are especially preferred for use herein. Chaperones of the hsp-60 class are structurally distinct from chaperones of the hsp-70 class. In particular, hsp-60 chaperones appear to form a stable scaffold of two heptamer rings stacked one atop another which interacts with partially folded elements of secondary structure. On the other hand, hsp-70 chaperones are monomers of dimers and appear to interact with short extended regions of a polypeptide.

Hsp70 chaperones are well conserved in sequence and function. Analogues of hsp-70 include the eukaryotic hsp70 homologue originally identified as the IgG heavy chain binding protein (BiP). BiP is located in all eukaryotic cells within the lumen of the endoplasmic reticulum (ER). The prokaryotic DnaK hsp70 protein chaperone in Escherichia coli shares about 50% sequence homology with an hsp70 KAR2 chaperone in yeast (Rose et al. 1989 Cell 57:1211-1221). Moreover, the presence of mouse BiP in yeast can functionally replace a lost yeast KAR2 gene (Normington et al. 19: 1223-1236).

Hsp-60 chaperones are universally conserved (Zeilstra-Ryalls et al., (1991) Ann. Rev. Microbiol. 45:301-325) and include hsp-60 homologues from large number of species, including man. They include, for example, the E. coli GroEL polypeptide; Ehrlichia sennetsu GroEL (Zhang et al., (1997) FEMS Immunol. Med. Microbiol. 18:39-46); Trichomonas vaginalis hsp-60 (Bozner et al, (1997) J. Parasitol. 83:224-229; rat hsp-60 (Venner et al., (1990) NAR 18:5309; and yeast hsp-60 (Johnson et al., (1989) Gene 84:295-302.

In a preferred aspect, the present invention relates to fragments of polypeptides of the hsp-60 family. These proteins being universally conserved, any member of the family may be used; however, in a particularly advantageous embodiment, fragments of GroEL, such as E. coli GroEL, are employed. It has also found that agarose-immobilised calmodulin does have a chaperoning activity, presumably because of its exposed hydrophobic groups.

The sequence of GroEL is available in the art and from academic databases (see GenBank Accession No. P06159); however, GroEL fragments which conform to the database sequence are inoperative. Specifically, the database contains a sequence in which positions 262 and 267 are occupied by Alanine and Isoleucine respectively. Fragments incorporating one or both of these residues at these positions are inoperative and unable to potentiate the folding of polypeptides. The invention, instead, relates to a GroEL polypeptide in which at least one of positions 262 and 267 is occupied by Leucine and Methionine respectively.

Derivative.

The present invention relates to derivatives of molecular chaperones, peptidyl-prolyl isomerases and thiolldisulphide oxidoreductases. In a preferred aspect, therefore, the terms “molecular chaperone”, “peptidyl-prolyl isomerase” and “thiol-disulphide oxidoreductase” include derivatives thereof which retain the stated activity. The derivatives provided by the present invention include splice variants encoded by mRNA generated by alternative splicing of a primary transcript, amino acid mutants, glycosylation variants and other covalent derivatives of molecular chaperones or foldases which retain the functional properties of molecular chaperones, peptidyl-prolyl isomerases and/or thiol/disulphide oxidoreductases. Exemplary derivatives include molecules which are covalently modified by substitution, chemical, enzymatic, or other appropriate means with a moiety other than a naturally occurring amino acid. Such a moiety may be a detectable moiety such as an enzyme or a radioisotope. Further included are naturally occurring variants of molecular chaperones or foldases found within a particular species, whether mammalian, other vertebrate, yeast, prokaryotic or otherwise.

Such a variant may be encoded by a related gene of the same gene family, by an allelic variant of a particular gene, or represent an alternative splicing variant of a molecular chaperone or foldase. Possible derivatives of the polypeptides employed in the invention are described below.

### DESCRIPTION OF PREFERRED EMBODIMENTS

Scaffold Proteins

In a preferred embodiment, the scaffold polypeptide is based on members of the cpn10/Hsp10 family, such as GroES or an analogue thereof. A highly preferred analogue is the T4 polypeptide Gp31. GroES analogues, including Gp31, possess a mobile loop (Hunt, J. F., et al., (1997) Cell 90, 361-371; Landry, S. J., et al., (1996) Proc. Natl. Acad. Sci. U.S.A. 93, 11622-11627) which may be inserted into, or replaced, in order to fuse the polypeptide to the scaffold.

Cpn10 homologues are widespread throughout animals, plants and bacteria. For example, a search of GenBank indicates that cpn10 homologues are known in the following species:

Actinobacillus actinomycetemcomitans; Actinobacillus pleuropneumoniae; Aeromonas salmonicida; Agrobacterium tumefaciens; Allochromatium vinosum; Amoeba proteus symbiotic bacterium; Aquifex aeolicus; Arabidopsis thaliana; Bacillus sp; Bacillus stearothermophilus; Bacillus subtilis; Bartonella henselae; Bordetella pertussis; Borrelia burgdorferi; Brucella abortus; Buchnera aphidicola; Burkholderia cepacia; Burkholderia vietnamiensis; Campylobacter jejuni; Caulobacter crescentus; Chiamydia muridarum; Chlamydia trachomatis; Chlamydophila pneumoniae; Clostridium acetobutylicum; Clostridium perfringens; Clostridium thermocellum; coliphage T; Cowdria ruminantium; Cyanelle Cyanophora paradoxa; Ehrlichia canis; Ehrlichia chaffeensis; Ehrlichia equi; Ehrlichia phagocytophila; Ehrlichia risticii; Ehrlichia sennetsu; Ehrlichia sp ‘HGE agent’; Enterobacter aerogenes; Enterobacter agglomerans; Enterobacter amnigenus; Enterobacter asburiae; Enterobacter gergoviae; Enterobacter intermedius; Erwinia aphidicola; Erwinia carotovora; Erwinia herbicola; Escherichia coli; Francisella tularensis; Glycine max; Haemophilus ducreyi; Haemophilus influenzae Rd; Helicobacter pylori; Holospora obtusa; Homo sapiens; Klebsiella ornithinolytica; Klebsiella oxytoca; Klebsiella planticola; Klebsiella pneumoniae; Lactobacillus helveticus; Lactobacillus zeae; Lactococcus lactis; Lawsonia intracellularis; Leptospira interrogans; Methylovorus sp strain SS; Mycobacterium avium; Mycobacterium avium subsp avium; Mycobacterium avium subsp paratuberculosis; Mycobacterium leprae; Mycobacterium tuberculosis; Mycoplasma genitalium; Mycoplasma pneumoniae; Myzus persicae primary endosymbiont; Neisseria gonorrhoeae; Oscillatoria sp NKBG; Pantoea ananas; Pasteurella multocida; Porphyromonas gingivalis; Pseudomonas aeruginosa; Pseudomonas aeruginosa; Pseudomonas putida; Rattus norvegicus; Rattus norvegicus; Rhizobium leguminosarum; Rhodobacter capsulatus; Rhodobacter sphaeroides; Rhodothermus marinus; Rickettsia prowazekii; Rickettsia rickettsii; Saccharomyces cerevisiae; Serratia ficaria; Serratia marcescens; Serratia rubidaea; Sinorhizobium meliloti; Sitophilus oryzae principal endosymbiont; Stenotrophomonas maltophilia; Streptococcus pneumoniae; Streptomyces albus; Streptomyces coelicolor; Streptomyces coelicolor; Streptomyces lividans; Synechococcus sp; Synechococcus vulcanus; Synechocystis sp; Thermoanaerobacter brockii; Thermotoga maritima; Thermus aquaticus; Treponema pallidum; Wolbachia sp; Zymomonas mobilis.

An advantage of cpn10 family subunits is that they possess a mobile loop, responsible for the protein folding activity of the natural chaperonin, which may be removed without affecting the scaffold.

Cpn10 with a deleted mobile loop possesses no biological activity, making it an advantageously inert scaffold, thus minimising any potentially deleterious effects. Insertion of an appropriate biologically active polypeptide can confer a biological activity on the novel polypeptide thus generated. Indeed, the biological activity of the inserted polypeptide may be improved by incorporation of the biologically active polypeptide into the scaffold.

Alternative sites for peptide insertion are possible. An advantageous option is in the position equivalent to the roof beta hairpin in GroES. This involves replacement of Glu-60 in Gp31 by the desired peptide. The amino acid sequence is Pro(59)-Glu(60)-Gly(61).

This is conveniently converted to a SmaI site at the DNA level (CCC:GGG) encoding Pro-Gly, leaving a blunt-ended restriction site for peptide insertion as a DNA fragment. Similarly, an insertion may be made at between positions 56 and 57 of the GroES sequence, and at equivalent positions in other cpn10 family members. Alternatively, inverse PCR may be used, to display the peptide on the opposite side of the scaffold.

Members of the cpn60/Hsp60 family of chaperonin molecules may also be used as scaffolds. For example, the tetradecameric bacterial chaperonin GroEL may be used. Advantageously, polypeptides would be inserted between positions 191 and 376, in particular between positions 197 and 333 (represented by SacII engineered and unique Cla I sites) to maintain intact the hinge region between the equatorial and the apical domains in order to impart mobility to the inserted polypeptide. The choice of scaffold may depend upon the intended application of the oligomer: for example, if the oligomer is intended for vaccination purposes (see below), the use of an immunogenic scaffold, such as that derived from Mycobacterium tuberculosis, is highly advantageous and confers an adjuvant effect.

Mutants of cpn60 molecules may also be used. For example, the single ring mutant of GroEL (GroELSR1) contains four point mutations which effect the major attachment between the two rings of GroEL (R452E, E461A, S463A and V464A) and is functionally inactive in vitro because it is release to bind GroES. GroELSR2 has an additional mutation at Glu191-Gly, which restores activity by reducing the affinity for GroES. Both of these mutants for ring structures and would be suitable for use as scaffolds.

Configurations of Oligomers According to the Invention

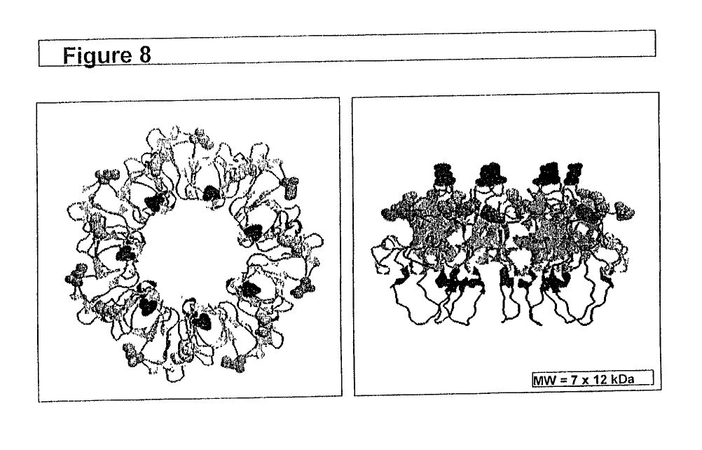

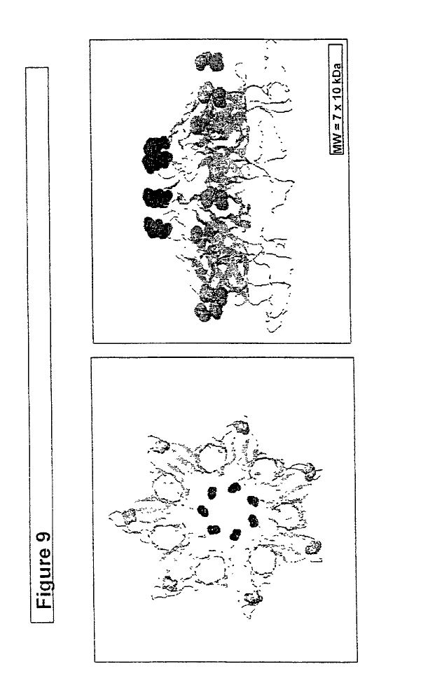

FIGS. 8-10 show various topologies and applications for scaffolded polypeptides in accordance with the present invention. In FIGS. 8 and 9, the possible insertion sites for polypeptides are shown. Insertion of polypeptides may be performed by any suitable technique, including those set forth by Doi and Yanagawa (FEBS Letters (1999) 457:1-4). As set forth therein, insertion of polypeptides may be combined with randomisation to produce libraries of polypeptide repertoires, suitable for display and selection.

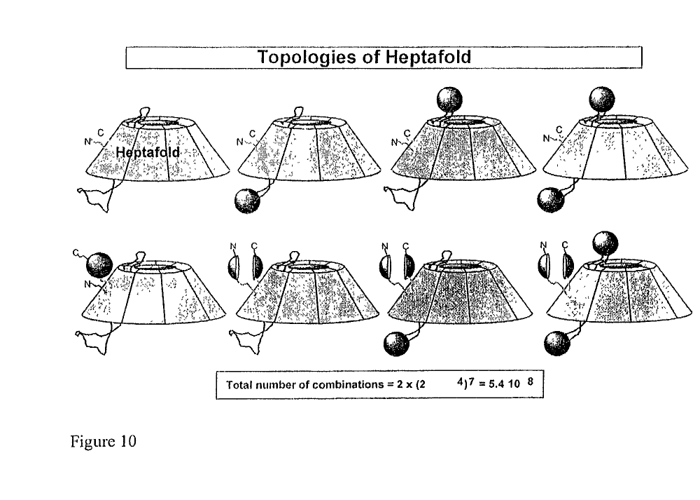

FIG. 10 illustrates the potential attachment sites for polypeptides to a circular scaffold, in this case Gp31. Reading from left to right, the figure shows: no attachment, attachment to the mobile loop, attachment to the roof β hairpin, attachment at both the mobile loop and the roof β hairpin, attachment at the C terminus, attachment at both N and C termini, attachment at both N and C termini and the mobile loop, and attachment at both N and C termini, the roof β hairpin and the mobile loop. As will be apparent, further configurations are possible, and can be combined in any way in the heptamer, leading to a total of 5.4×10⁸ possible configurations.

Recombinant DNA Techniques

The present invention advantageously makes use of recombinant DNA technology in order to construct polypeptide monomers and oligomers. Advantageously, polypeptide monomers or oligomers may be expressed from nucleic acid sequences which encode them.

As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2m plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells which have been transformed, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the polypeptide according to the invention. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the heterologous nucleic acid coding sequence. Such a promoter may be inducible or constitutive. The promoters are operably linked to the coding sequence by inserting the isolated promoter sequence into the vector. Many heterologous promoters may be used to direct amplification and/or expression of the coding sequence. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to the coding sequence. using linkers or adapters to supply any required restriction sites. Promoters for use in bacterial systems will also generally contain a Shine-Delgarno sequence operably linked to the coding sequence.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int- phage such as the CE6 phage which is commercially available (Novagen, Madison, USA). other vectors include vectors containing the lambda PL promoter such as PLEX (Invitrogen, NL), vectors containing the trc promoters such as pTrcHisXpressTM (Invitrogen) or pTrc99 (Pharmacia Biotech, SE), or vectors containing the tac promoter such as pKK223-3 (Pharmacia Biotech) or PMAL (new England Biolabs, MA, USA).

Moreover, the coding sequence according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, provided such promoters are compatible with the host cell systems.

Transcription of a coding sequence by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the coding sequence, but is preferably located at a site 5′ from the promoter.

Advantageously, a eukaryotic expression vector may comprise a locus control region (LCR). LCRs are capable of directing high-level integration site independent expression of transgenes integrated into host cell chromatin, which is of importance especially where the coding sequence is to be expressed in the context of a permanently-transfected eukaryotic cell line in which chromosomal integration of the vector has occurred, in vectors designed for gene therapy applications or in transgenic animals.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA.

An expression vector includes any vector capable of expressing nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, nucleic acids may be inserted into a vector suitable for expression of cDNAs in mammalian cells, e.g. a CMV enhancer-based vector such as pEVRF (Matthias, et al., (1989) NAR 17, 6418).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

### DESCRIPTION OF PREFERRED EMBODIMENTS

In a preferred aspect, the present invention relates to a method for oligomerising polypeptides and novel oligomeric polypeptides producible thereby. By incorporating polypeptides into a scaffold which is capable of oligomerising, as described herein, oligomers may be produced wherein the chosen polypeptides are juxtaposed. The polypeptides selected for oligomerisation may be the same or different; thus, it is possible to produce homooligomers or heterooligomers.

Preferably, the oligomeric proteins of the invention are oligomeric minichaperone polypeptides. Oligomeric minichaperones have been shown the possess particularly advantageous properties.

The invention thus relates to the use of oligomeric molecular chaperone fragments, whether alone or in combination with other polypeptides, for potentiating the folding or refolding of polypeptides.

The present invention may be practised in a number of configurations, according to the required use to which the invention is to be put. In a first configuration, the invention relates to the use of minichaperone oligomers alone to potentiate the folding or refolding of polypeptides. This may be performed in vivo or in vitro, in solution or on a solid support. For example, oligomerised minichaperones may be immobilised onto resins and packed into columns for use in refolding polypeptides which are passed through the column. Methods for immobilising minichaperones are described in International patent application WO99/05163, incorporated herein by reference.

In an alternative configuration, minichaperone oligomers according to the invention may be expressed in vivo or administered to cells or organisms in vivo in order to potentiate protein folding therein.

In a second configuration, the invention provides a combination of a molecular chaperone and a thiol/disulphide oxidoreductase to facilitate protein folding. One or both of the chaperones and thiol/disulphide oxidoreductases may be incorporated into the scaffold according to the invention. The combination of a molecular chaperone and a thiol/disulphide oxidoreductase provides a synergistic effect on protein folding which results in a greater quantity of active, correctly folded protein being produced than would be expected from a merely additive relationship. Advantageously, one or more of the components used to potentiate protein folding in accordance with the present invention is immobilised on a solid support. However, both molecular chaperones and thiol/disulphide oxidoreductases may be used in solution. They may be used in free solution, but also in suspension, for example bound to a matrix such as beads, for example Sepharose beads, or bound to solid surfaces which are in contact with solutions, such as the inside surfaces of bottles containing solutions, test tubes and the like.

In a third configuration, the invention relates a to the use of a combination of a molecular chaperone and a thiol/disulphide oxidoreductase with a peptidyl prolyl isomerase. The peptidyl prolyl isomerase may be present either incorporated into the oligomeric scaffold, bound to a solid support, or in solution. Moreover, it may be bound to beads suspended in solution. The peptidyl prolyl isomerases may be used together with a molecular chaperone alone, with a thiol/disulphide oxidoreductase alone, or with both a molecular chaperone and a thiol/disulphide oxidoreductase. In the latter case, further synergistic effects are apparent over the additive effects which would be expected from the use of the three components together. In particular, an increase in the proportion of the folded protein which is recovered as monodisperse protein, as opposed to aggregated protein, increases substantially.

Used in accordance with any of the foregoing configurations, or otherwise in accordance with the following claims, the invention may be used to facilitate protein folding in a variety of situations. For example, the invention may be the used to assist in refolding recombinantly produced polypeptides, which are obtained in an unfolded or misfolded form. Thus, recombinantly produced polypeptides may be passed down a column on which is immobilised a composition comprising protein disulphide isomerase and/or a molecular chaperone and/or a prolyl peptidyl isomerase.

In an alternative embodiment, in a the invention may be employed to maintain the folded conformation of proteins, for example during storage, in order to increase shelf life. under storage conditions, many proteins lose their activity, as a result of disruption of correct folding. The presence of molecular chaperones, in combination with foldases, reduces or reverses the tendency of polypeptides to become unfolded and thus greatly increases the shelf life thereof. In this embodiment, the invention may be applied to reagents which comprise polypeptide components, such as enzymes, tissue culture components, and other proteinaceous reagents stored in solution.

In a further embodiment, the invention may be used to potentiate the correct folding of proteins which, through storage, exposure to denaturing conditions or otherwise, have become misfolded. Thus, the invention may be used to recondition reagents or other proteins. For example, proteins in need of reconditioning may be passed down a column to which is immobilised a combination of reagents in accordance with he invention. Alternatively, beads having immobilised thereon such a combination may be suspended in a solution comprising the proteins in need of reconditioning. Moreover, the components of the combination according to the invention may be added in solution to the proteins in need of reconditioning.

As noted above, the components of the combination according to the invention may comprise derivatives of molecular chaperones or foldases, including variants of such polypeptides which retain common structural features thereof. Variants which retain common structural features can be fragments of molecular chaperones or foldases. Fragments of molecular chaperones or foldases comprise smaller polypeptides derived from therefrom. Preferably, smaller polypeptides derived from the molecular chaperones or foldases according to the invention define a single feature which is characteristic of the molecular chaperones or foldases. Fragments may in theory be almost any size, as long as they retain the activity of the molecular chaperones or foldases described herein.

With respect to molecular chaperones of the GroEL/hsp-60 family, a preferred set of fragments have been identified which possess the desired activity. These fragments are set forth in our copending international patent application PCT/GB96/02980 and in essence comprise any fragment comprising at least amino acid residues 230-271 of intact GroEL, or their equivalent in another hsp-60 chaperone. Preferably, the fragments should not extend beyond residues 150-455 or 151-456 of GroEL or their equivalent in another hsp-60 chaperone. Where the fragments are GroEL fragments, they must not possess the mutant GroEL sequence as set forth above; in other words, they must not have an Alanine residue at position 262 and/or an Isoleucine residue at position 267 of the sequence of intact GroEL.

Advantageously, the fragments comprise the apical domain of GroEL, or its equivalent in other molecular chaperones, or a region homologous thereto as defined herein. The apical domain spans amino acids 191-376 of intact GroEL. This domain is found to be homologous amongst a wide number of species and chaperone types.

Preferably, molecular chaperones according to the invention are homologous to, or are capable of hybridising under stringent conditions with, a region corresponding to the apical domain of GroEL as defined above.

In a highly preferred embodiment, the fragments are selected from the group consisting of residues 191-376, 191-345 and 191-335 of the sequence of intact GroEL.

Derivatives of the molecular chaperones or foldases also comprise mutants thereof, including mutants of fragments and other derivatives, which may contain amino acid deletions, additions or substitutions, subject to the requirement to maintain the activity of the molecular chaperones or foldases described herein. Thus, conservative amino acid substitutions may be made substantially without altering the nature of the molecular chaperones or foldases, as may truncations from the 5′ or 3′ ends. Deletions and substitutions may moreover be made to the fragments of the molecular chaperones or foldases comprised by the invention. Mutants may be produced from a DNA encoding a molecular chaperone or foldase which has been subjected to in vitro mutagenesis resulting e.g. in an addition, exchange and/or deletion of one or more amino acids. For example, substitutional, deletional or insertional variants of molecular chaperones or foldases can be prepared by recombinant methods and screened for immuno-crossreactivity with the native forms of the relevant molecular chaperone or foldase.

The fragments, mutants and other derivative of the molecular chaperones or foldases preferably retain substantial homology with the native molecular chaperones or foldases. As used herein, “homology” means that the two entities share sufficient characteristics for the skilled person to determine that they are similar in origin and function. Preferably, homology is used to refer to sequence identity. Thus, the derivatives of molecular chaperones or foldases preferably retain substantial sequence identity with native forms of the relevant molecular chaperone or foldase.

In the context of the present invention, a homologous sequence is taken to include an amino acid sequence which is at least 60, 70, 80 or 90% identical, preferably at least 95 or 98% identical at the amino acid level over at least 20, preferably 30 amino of the minichaperone. In particular, homology should typically be considered with respect to those regions of the sequence known to be essential for the chaperone activity rather than non-essential neighbouring sequences. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see http://www.ncbi.nih.gov/BLAST/), FASTA (Atschul et al., 1990, J. Mol. Biol., 403410; FASTA is available for online searching at, for example, http://www.2.ebi.ac.uk.fasta3) and the GENEWORKS suite of comparison tools. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Alternatively, sequence similarity may be defined according to the ability to hybridise to a complementary strand of a chaperone or foldase sequence as set forth above.

Preferably, the sequences are able to hybridise—with high stringency. Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6× SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1× SSC, 0.1% SDS.

Moderate stringency refers to conditions equivalent to hybridisation in the above described solution but at about 60-62° C. In that case the final wash is performed at the hybridisation temperature in 1× SSC, 0.1% SDS.

Low stringency refers to conditions equivalent to hybridisation in the above described solution at about 50-52° C. In that case, the final wash is performed at the hybridisation temperature in 2× SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the probe also play a role.

The invention also envisages the administration of polypeptide oligomers according to the invention as compositions, preferably for the treatment of diseases associated with protein misfolding. The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the combination by other than parenteral administration, it will be coated by, or administered with, a material to prevent its inactivation. For example, the combination may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the combination of polypeptides is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the combination of the invention as hereinbefore defined for use in the treatment of disease. Consequently there is provided the use of a combination of the invention for the manufacture of a medicament for the treatment of disease associated with aberrant protein/polypeptide structure. The aberrant nature of the protein/polypeptide may be due to misfolding or unfolding which in turn may be due to an anomalous e.g. mutated amino acid sequence. The protein/polypeptide may be destabilised or deposited as plaques e.g. as in Alzheimer's disease. The disease might be caused by a prion. A polypeptide-based medicament of the invention would act to renature or resolubilise aberrant, defective or deposited proteins.

The invention is further described below, for the purposes of illustration only, in the following examples.

### EXAMPLES

1. General Experimental Procedures

Bacterial and Bacteriophage Strains.

The E. Coli strains used in this study were: C41(DE3), a mutant of BL21(DE3) capable of expressing toxic genes (Miroux, B. & Walker, J. E. (1996) J. Mol. Biol. 260, 289-298); SV2 (B178groEL44), SV3 (B178groEL59) and SV6 (B178groEL673): isogenic strains carrying temperature-sensitive alleles of groEL; SV1(=B178) (Georgopoulos, C., Hendrix, R. W., Casjens, S. R. & Kaiser, A. D. (1973) J. Mol. Biol. 76, 45-60), A190 (ΔgroEL::kan^{R}) [pBAD-EL] (Ivic, A., Olden, D., Wallington, E. J. & Lund, P. A. (1997) Gene 194, 1-8), and TG1 (Gibson, T. J. (1984) Ph.D. thesis, University of Cambridge, U.K). Bacteriophage λb₂cI (Zeilstra-Ryalls, J., Fayet, O., Baird, L. & Georgopoulos, C. (1993) J. Bacteriol. 175, 1134-1143) was used according to standard methods (Arber, W., Enquist, L., Hohn, B., Murray, N. E. & Murray, K. (1983) in Lambda II., ed. R. W. Hendrix, J. W. r., F. W. Stahl and R. A. Weisberg (Cold Spring Harbor Laboratory, Cold Spring Harbor, N.Y.), pp. 433-466); plaque formation was assayed at 30° C. T4D0, a derivative of bacteriophage T4 (Zeilstra-Ryalls, J., Fayet, O., Baird, L. & Georgopoulos, C. (1993) J. Bacteriol. 175, 1134-1143), was used according to standard methods (Karam, J. D. (1994) Molecular biology of bacteriophage T4. (American Society for Microbiology, Washington, D.C.)); plaque formation was assayed at 37° C.

Plasmid Constructions.

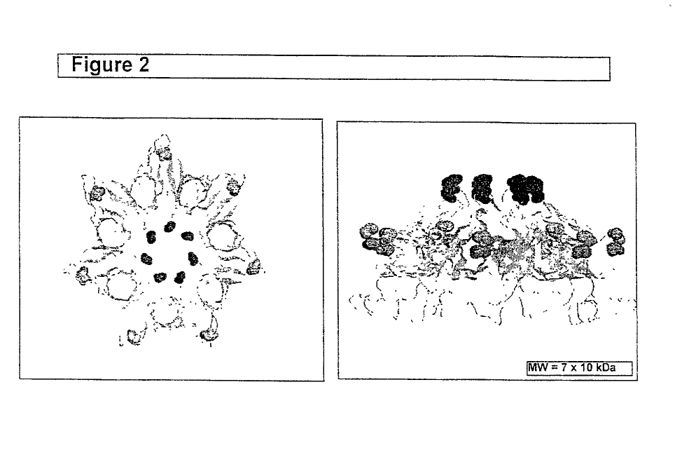

Standard molecular biology procedures were used (Sambrook, J., Fritsch, E. F. & Maniatis, T. (1989) Molecular Cloning. A Laboratory Manual (Cold Spring Harbor Laboratory Press, N.Y.)). The schematic organisation of the plasmids used in this study is represented FIG. 2. Gp31 gene was PCR (polymerase chain reaction) amplified using two oligonucleotides 5′-C TTC AGA CAT ATG TCT GAA GTA CAA CAG CTA CC-3′ and 5′-TAA CGG CCG TTA CTT ATA AAG ACA CGG AAT AGC-3′ producing a 358 bp DNA using pSV25 (van der Vies, S., Gatenby, A. & Georgopoulos, C. (1994) Nature 368, 654-656) as template. The DNA sequence of a part of the mobile loop of Gp31 (residues 25 to 43) was removed by PCR, as described (Hemsley, A., Arnheim, N., Toney, M. D., Cortopassi, G. & Galas, D. J. (1989) Nucleic Acids Res. 17, 6545-6551), using oligonucleotides 5′-GGA GAA GTT CCT GAA CTG-3′ and 5′-GGA TCC GGC TTG TGC AGG TTC-3′, creating a unique BamHI site (bold characters). GroEL gene minichaperone (corresponding to the apical domain of GroEL, residues 191 to 376; (Zahn, R., Buckle, A. M., Perret, S., Johnson, C. M. J., Corrales, F. J., Golbik, R. & Fersht, A. R. (1996) Proc. Natl. Acad. Sci. U.S.A. 93, 15024-15029)) was amplified by PCR using oligonucleotides, containing a BamHI site (underlined), 5′-TTC GGA TCC GAA GGT ATG CAG TTC GAC C-3′ and 5′-GTT GGA TCC AAC GCC GCC TGC CAG TTT C-3′ and cloned into the unique BamHI site of pRSETA-Gp31Δloop vector, inserting minichaperone GroEL(191-376) in frame into Gp31Δloop sequence. The single ring GroEL_{SR1} mutant contains four amino acid substitutions (R452E, E461A, S463A, and V464A) into the equatorial interface of GroEL, which prevent the formation of double rings (Weissman, J. S., Hohl, C. M., Kovalenko, O., Kashi, Y., Chen, S., Braig, K., Saibil, H. R., Fenton, W. A. & Horwich, A. L. (1995) Cell 83, 577-587). The corresponding mutations were introduced into groEL by PCR (Hemsley, A., Arnheim, N., Toney, M. D., Cortopassi, G. & Galas, D. J. (1989) Nucleic Acids Res. 17, 6545-6551) using oligonucleotides 5′-TGA GTA CGA TCT GTT CCA GCG GAG CTT CC-3′ and 5′-ATT GCG GCG AAG CGC CGG CTG CTG TTG CTA ACA CCG-3′ and pRSETA-Eag I GroEL or GroESL vectors (Chatellier, J., Hill, F., Lund, P. & Fersht, A. R. (1998) Proc. Natl. Acad. Sci. U.S.A. 95, 9861-9866) as template; silent mutations, in respect to the codon usage in E. coli, create a unique Mfe I (bold characters) and Nae I (underlined). GroEL(E191G; groEL44 allele) gene was PCR amplified from E. coli SV2 strain (Zeilstra-Ryalls, J., Fayet, O., Baird, L. & Georgopoulos, C. (1993) J. Bacteriol. 175, 1134-1143) using two oligonucleotides 5′-T AGC TGC CAT ATG GCA GCT AAA GAC GTA AAA TTC GG-3′ and 5′-ATG TAA CGG CCG TTA CAT CAT GCC GCC CAT GCC ACC-3′ producing a 1,659 bp DNA with unique sites for Nde I and Eag I (underlined). The different genes were subcloned into the unique Nde I and Eag I unique sites of pACYC184, pJC and pBAD30 (Guzman, L. -M., Belin, D., Carson, M. J. & Beckwith, J. (1995) J. Bacteriol. 177, 4121-4130) vectors (Chatellier, J., Hill, F., Lund, P. & Fersht, A. R. (1998) Proc. Natl. Acad. Sci. U.S.A. 95, 9861-9866). A colony-based PCR procedure was used to identify the positive clones (Chatellier, J., Mazza, A., Brousseau, R. & Vernet, T. (1995) Analyt. Biochem. 229, 282-290). PCR cycle sequencing using fluorescent dideoxy chain terminators (Applied Biosystems) were performed and analysed on an Applied Biosystems 373A Automated DNA. All PCR amplified DNA fragments were sequenced after cloning.

Proteins Expression Purification and Characterisation. The GroE proteins, ˜57.5 kDa GroEL and ˜10 kDa GroES, were expressed and purified as previously described (Chatellier, J., Hill, F., Lund, P. & Fersht, A. R. (1998) Proc. Natl. Acad. Sci. U.S.A. 95, 9861-9866; Corrales, F. J. & Fersht, A. R. (1996) Folding & Design 1,265-273). GroEL_{SR1} mutant was expressed and purified using the same procedure used for the wild-type GroEL; GroEL_{SR1} mutant was separated from emdogenous wild-type GroEL by ammonium sulphate precipitation at 30% saturation. GroEL(E191G) protein was expressed by inducing the P_{BAD} promoter of pBAD30 based vector with 0.2% arabinose in E. coli SV2 strain (Zeilstra-Ryalls, J., Fayet, O., Baird, L. & Georgopoulos, C. (1993) J. Bacteriol. 175, 1134-1143). Purification was performed essentially as described (Corrales, F. J. & Fersht, A. R. (1996) Folding & Design 1,265-273). Residual peptides bound to GroEL proteins were removed by ion-exchange chromatography on a MonoQ column (Pharmacia Biotech.) in presence of 25% methanol. The over-expression of histidine-tagged (short histidine tail; sht)-minichaperone GroEL(191-376) in E. coli C41(DE3) cells and, the purification and the removal of sht by thrombin cleavage were carried out essentially as previously described (Zahn, R., Buckle, A. M., Perret, S., Johnson, C. M. J., Corrales, F. J., Golbik, R. & Fersht, A. R. (1996) Proc. Natl. Acad. Sci. U.S.A. 93, 15024-15029). Gp31 proteins wild-type (˜12 kDa), Δloop (˜10.4 kDa) and MC₇ (˜30.6 kDa), were expressed by inducing the T7 promoter of pRSETA-Eag I based vectors with isopropyl-β-D-thiogalactoside (IPTG) in E. coli C41(DE3) (Miroux, B. & Walker, J. E. (1996) J. Mol. Biol. 260, 289-298) overnight at 25° C. Purification procedures were essentially as described (van der Vies, S., Gatenby, A. & Georgopoulos, C. (1994) Nature 368, 654-656; Castillo, C. J. & Black, L. W. (1978) J. Biol. Chem. 253, 2132-2139). Ammonium sulphate precipitation (only 20% saturation for Δloop; 30 to 70% saturation for wild-type and MC₇) was followed by ion-exchange chromatography on a DEAE-Sepharose column (Pharmacia Biotech.). Gp31 proteins were eluted with a 0-0.5 M NaCl gradient in 20 mM Tris-HCl, 1 mM EDTA, 1 mM β-mercaptoethanol, pH 7.5; Δloop and MC₇ eluted between 0.32-0.44 and 0.38-0.48 mM NaCl, respectively. Gp31 proteins were further purified by gel filtration chromatography on a Superdex™ 200 (Hiload 26/10) column (Pharmacia Biotech.) equilibrated with 100 mM Tris-HCl, pH 7.5 and, dialysed against and stored in 50 mM Tris-HCl, 0.1 mM EDTA, 1 mM β-mercaptoethanol, pH 7.5. Proteins were analysed by electrospray mass spectrometry. Protein concentration was determined by absorbance at 276 nm using the method of Gill & von Hippel (Gill, S.C. & von Hippel, P. H. (1989) Analyt. Biochem. 182, 319-326) and confirmed by quantitative amino acid analysis.

Constitutive expression under the control of the tetracycline-resistance gene promoter/operator was obtained using the high copy-number pJC vectors (Chatellier, J., Hill, F., Lund, P. & Fersht, A. R. (1998) Proc. Natl. Acad. Sci. U.S.A. 95, 9861-9866). pBAD30 vector allows inducible expression with 0.2-0.5% arabinose controlled by the P_{BAD} promoter and its regulatory gene, araC (Guzman, L.-M., Belin, D., Carson, M. J. & Beckwith, J. (1995) J. Bacteriol. 177, 41214130). The level of expression of MC₇ was analysed by 15% sodium dodecyl sulphate polyacrylamide gel electrophoresis (SDS-PAGE) under non-reducing conditions followed by Western blotting as described (Chatellier, J., Hill, F., Lund, P. & Fersht, A. R. (1998) Proc. Natl. Acad. Sci. U.S.A. 95, 9861-9866).

Molecular Weight Determination by Analytical Gel Filtration Chromatography and Analytical Ultracentrifugation.

One hundred μl aliquots of protein (1 mg.mL⁻¹) were loaded onto a Superdex™ 200 (HR 10/30) column (Pharmacia Biotech.) equilibrated with 50 mM Tris-HCl, 150 mM NaCl, pH 7.5 at 0.5 mL.min⁻¹ at 20° C. The column was calibrated using gel filtration standards from Pharmacia Biotech. (thyroglobulin, MW=669 kDa; ferritin, MW-440 kDa; aldolase, MW=158 kDa; ovalbulmin, MW=45 kDa; chymotrypsinogen MW=25 kDa; RNase, MW=13 kDa). Molecular weights were determined by logarithmic interpolation.

Sedimentation analysis was performed in 50 mM Tris-HCl, 2.5 mM DTE (dithioerythritol), pH 7.2 at 20° C. with protein concentration in the range 45-300 μM, scanning at 280 nm, with a Beckman XL-A analytical ultracentrifuge, using an An-60Ti rotor. Sedimentation equilibrium experiments were at 10,000 rev.min⁻¹ with overspeeding at 15,000 rev.min⁻¹ for 6 hours to speed the attainment of equilibrium. Scans were taken at intervals of 24 hours, until successive scans superimposed exactly, when the later scan was taken as being operationally at equilibrium. To evaluate the apparent average molecular weight, data were fitted by non-linear regression.

Circular Dichroism Spectroscopy (CD).

Far UV (200-250 nm)-CD spectra at 25° C. were measured on a Jasco J720 spectropolarimeter interfaced with a Neslab PTC-348WI water bath, using a thermostatted cuvette of 0.1 cm path length. Spectra are averages of 10 scans and were recorded with a sampling interval of 0.1 nm. Thermal denaturation was carried out from 5-95° C. at a linear rate of 1° C.min⁻¹ and monitored at 222 nm. The reversibility was checked after incubation at 95° C. for 20 min and cooling to and equilibration at 5° C. The protein concentration was 45 μM in 10 mM sodium phosphate buffer pH 7.8, 2.5 mM DTE (dithioerythrol).

GroES Binding and Competition Assays by ELISA (Enzyme-Linked Immunosorbant Assay).

Proteins were coated onto plastic microtitre plates (Maxisorb, Nunc) overnight at 4° C. at a concentration of 10 μg/mL in carbonate buffer (50 mM NaHCO₃, pH 9.6). Plates were blocked for 1 hour at 25° C. with 2% Marvel in PBS (phosphate buffered saline: 25 mM NaH₂PO₄, 125 mM NaCl, pH 7.0). GroES, at 10 μg/mL in 100 μL of 10 mM Tris-HCl, 200 mM KCl, pH 7.4, were bound at 25° C. for 1 hour. Bound GroES were detected with rabbit anti-GroES antibodies (Sigma) followed by anti-rabbit immunoglobulins horseradish peroxidase conjugated antibodies (Sigma).

A peptide corresponding to the mobile loop of GroES (residues 16 to 32, numbered as in Hemmingsen, S. M., Woolford, C., van, d. V. S., Tilly, K., Dennis, D. T., Georgopoulos, C. P., Hendrix, R. W. & Ellis, R. J. (1988) Nature 333, 330-334) was synthesised as described (Chatellier, J., Buckle, A. M. & Fersht, A. R. (1999) J. Mol. Biol., in press). The inhibition of the binding of MC₇ proteins by the free peptide was analysed by ELISA, essentially as above, by adding different concentrations (between 10,000 to 0.1 μM) of free peptide solved in 0.1% TFA solution to 1 μg of proteins prior incubation to coated GroES proteins (10 μg/mL). GroEL molecules were detected with rabbit anti-GroEL antibodies (Sigma) followed by anti-rabbit immunoglobulins horseradish peroxidase conjugate antibodies (Sigma). ELISAs were developed with 3′,3′,5′,5′-tetramethylbenzidine (TMB, Boehringer Mannheim). Reactions were stopped with 50 μl of 1M H₂SO₄ after 10 min and readings taken by subtracting the O.D._{650 nm} from the O.D._{450 nm}.

Anti-GroEL Antibodies Binding by ELISA.

The same amount of proteins (1 μg) were coated as described above. GroEL molecules were detected with either (i) rabbit anti-GroEL horseradish peroxidase conjugate antibodies (9 mg/mL; Sigma) or (ii) rabbit anti-GroEL antibodies (11.5 mg/mL; Sigma) followed by anti-rabbit immunoglobulins horseradish peroxidase conjugate antibodies (Sigma). ELISAs were developed as described above.

In vitro Refolding Experiments.

Refolding assays of pig heart mitochondrial malate dehydrogenase (mtMDH; Boehringer-Mannheim) and aggregation protection were carried out essentially as described (Peres Ben-Zvi, A. P., Chatellier, J., Fersht, A. R. & Goloubinoff, P. (1998) Proc. Natl. Acad. Sci. U.S.A. 95, 15275-15280). The concentrations of MC₇ used were between 8-16 μM (reporter to protomer).

In vivo Complementation Experiments.

Complementation experiments were performed by transforming electro-competent SV2 or SV6 cells with the pJC series of expression vectors and plating an aliquot of the transformation reactions directly at 43° C. The percentage of viable cells relative to the growth at 30° C. was determined. A representative number of clones which grew at 43° C. were incubated in absence of any selective markers at permissive temperature. After prolonged growth the loss of the pJC plasmids and the ts phenotype were verified. Each experiment was performed in duplicate. Plasmids carrying no groE genes or encoding the GroE proteins were used as negative or positive controls, respectively.

P1 transduction (Miller, J. H. (1972) Experiments in Molecular Genetics (Cold Spring Harbor, N.Y.)), using strain AI90 (ΔgroEL::kan^{R}) [pBAD-EL] as donor (Ivic, A., Olden, D., Wallington, E. J. & Lund, P. A. (1997) Gene 194, 1-8), was used to delete the groEL gene of TG1 cells transfected by the different pJC vectors. Transductants were selected on LB plates containing 10 μg/mL of kanamycin at 37° C. Approximately 25 colonies were transferred onto plates containing kanamycin at 50 μg/mL. After incubation for 24 h at 37° C., colonies that grew were screened by PCR as described.

AI90 (ΔgroEL::kan^{R}) [pBAD-EL] cells were transformed with the pJC vector series. Transformants were selected at 37° C. on LB supplemented with 50 μg.ml⁻¹ of kanamycin, 120 μg.mL⁻¹ of ampicillin, 25 μg.mL⁻¹ of chloramphenicol and 0.2% L(+)arabinose. Depletion of GroEL protein was analysed at 37° C. by plating the same quantity of AI90 [PBAD-EL+pJC vectors] cells on LB plates containing 1% D(+)glucose or various amount of arabinose.

Each experiment was performed in triplicate. Plasmids carrying no groE genes or encoding the GroE proteins were used as negative or positive controls, respectively.

Effect on Lorist6 Replication of Over-Expressing of MC₇.

The effect of over-expressing Gp31 proteins from pJC vector series on the replication of the bacteriophage λ origin vector, Lorist6 (Gibson, T. J., Rosenthal, A. & Waterston, R. H. (1987) Gene 53, 283-286) in TG1 (Gibson, T. J. (1984) Ph.D. thesis, University of Cambridge, U.K) or SV1 (Georgopoulos, C., Hendrix, R. W., Casjens, S. R. & Kaiser, A. D. (1973) J. Mol. Biol. 76, 45-60) cells was determined essentially as described (Chatellier, J., Hill, F., Lund, P. & Fersht, A. R. (1998) Proc. Natl. Acad. Sci. U.S.A. 95, 9861-9866).

### Example 1

### Gp31 Protein as a Scaffold for Displaying Heptameric GroEL Minichaperone.

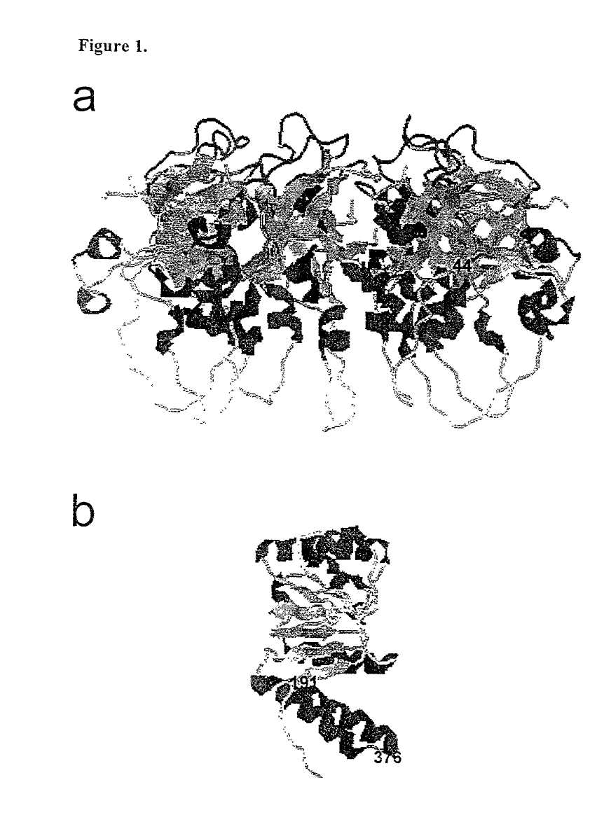

We describe a scaffold on which any polypeptide may be hung; as a result, the polypeptide is oligomerised. The scaffold is the bacteriophage T4 Gp31 (gene product) heptamer. The monomeric protein is 12 kDa, but it spontaneously forms a stable heptameric structure (90 kDa) of which the three-dimensional structure is known from X-ray crystallography (Hunt, J. F., van der Vies, S., Henry, L. & Deisenhofer, J. (1997) Cell 90, 361-371). This illustrates that a highly mobile polypeptide loop (residues 25 to 43; Chatellier, J., Mazza, A., Brousseau, R. & Vernet, T. (1995) Analyt. Biochem. 229, 282-290) projects from each subunit (FIG. 1). The basis of the method is the substitution of this loop by a chosen peptide sequence.

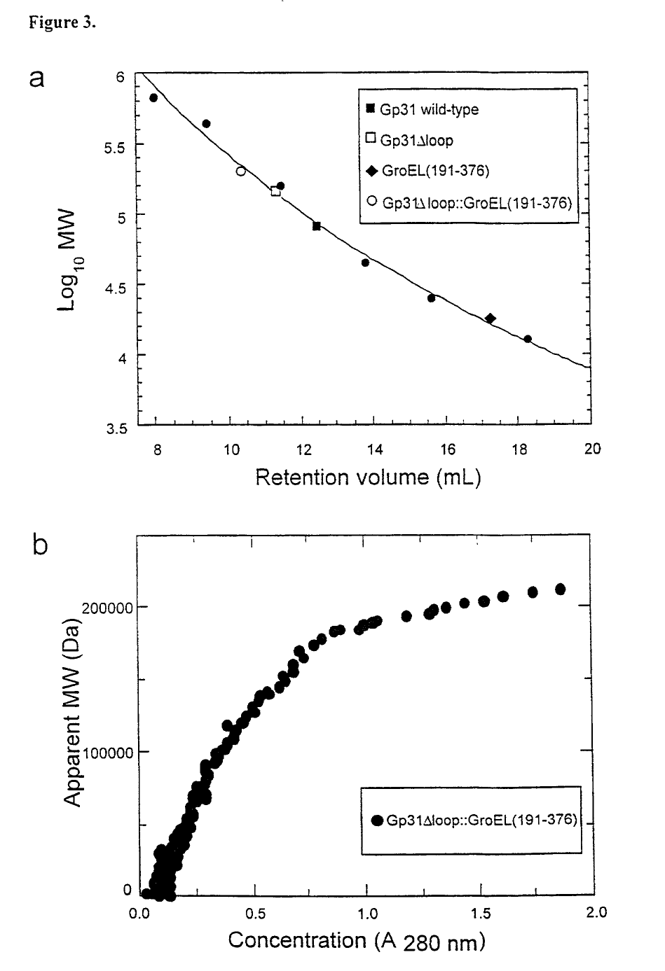

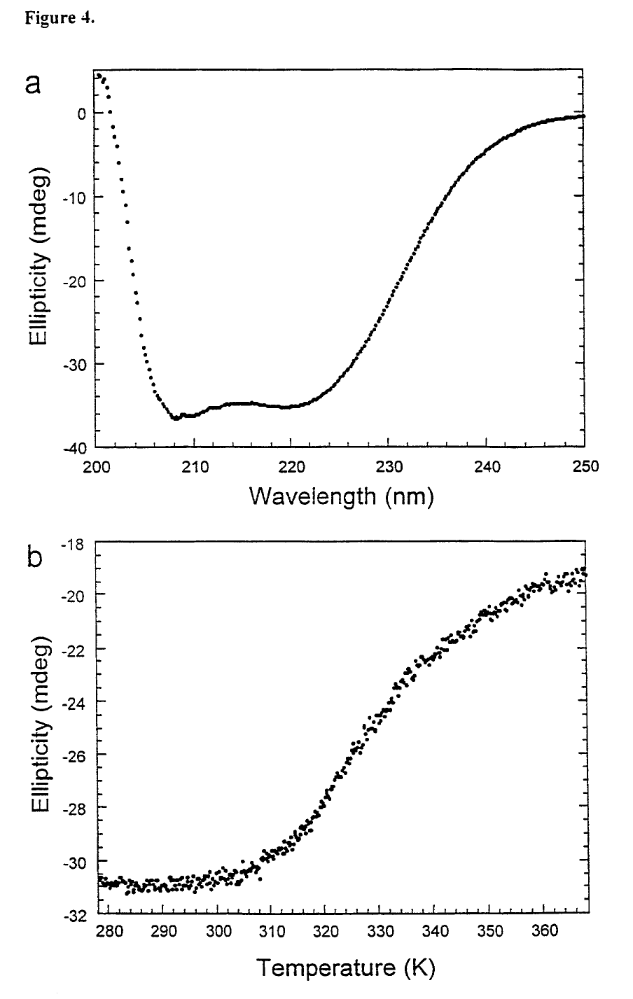

In an effort to increase the avidity of minichaperones for substrates, and consequently to improve their chaperonin-facilitated protein folding, we generated the fusion protein, Gp31Δloop::GroEL(191-376) (hereafter named MC₇), where the mobile loop of Gp31 was replaced by the sequence of minichaperone GroEL (residues 191 to 376) (FIG. 2). MC₇ was cloned downstream of the T7 promoter of pRSETAsht-Eag I vector (Chatellier, J., Hill, F., Lund, P. & Fersht, A. R. (1998) Proc. Natl. Acad. Sci. U.S.A. 95, 9861-9866). After sonication, the soluble and insoluble fractions of IPTG-induced transfected C41(DE3) cells (Miroux, B. & Walker, J. E. (1996) J. Mol. Biol. 260, 289-298) were analysed by SDS-PAGE. Most of MC₇ was present in the insoluble fraction. Insoluble material dissolved in 8 M urea was efficiently refolded by dialysis at 4° C. MC₇ was purified by ion-exchange and gel filtration chromatography. MC₇ was over-expressed in C41(DE3) cells to give 0.25-0.5 g purified protein per L of culture. Purified MC₇ coincided to seven 30.6 kDa subunits of Gp31Δloop::GroEL(191-376) as determined by analytical size exclusion chromatography (FIG. 3a) and analytical ultracentrifugation (FIG. 3b); Gp31Δloop corresponds to a tetra-decamer (14 subunits). The introduction of a foreign polypeptide in the Gp31 scaffold does do prevent its oligomerisation ability. The electronic microscopy studies of MC₇ revealed views that correspond to front views of oligomers with a diameter close to the one of GroEL (J. L. Carrascosa, J. C. & A. R. F., unpublished). The circular dichroism spectrum of MC₇ indicated significant a-helical structure (FIG. 4a). The thermal unfolding monitored by far UV-CD was reversible although more than one transition exist (FIG. 4b).

Bacterial GroES or the human mitchodrial Hsp10 homologous oligomerisable scaffolds have been also successfully used to oligomerise polypeptides displayed in their mobile loops¹. ¹ Please send me details of these experiments.

### Example 2

### Binding to Heptameric Bacterial Co-Chaperonin, GroES.

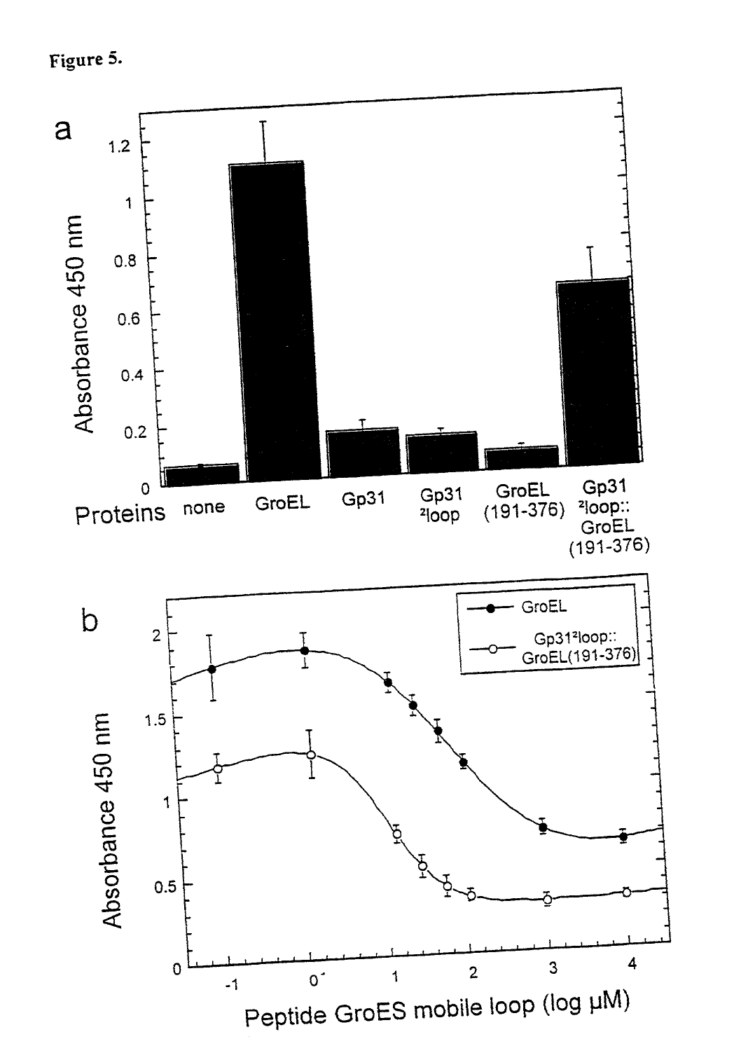

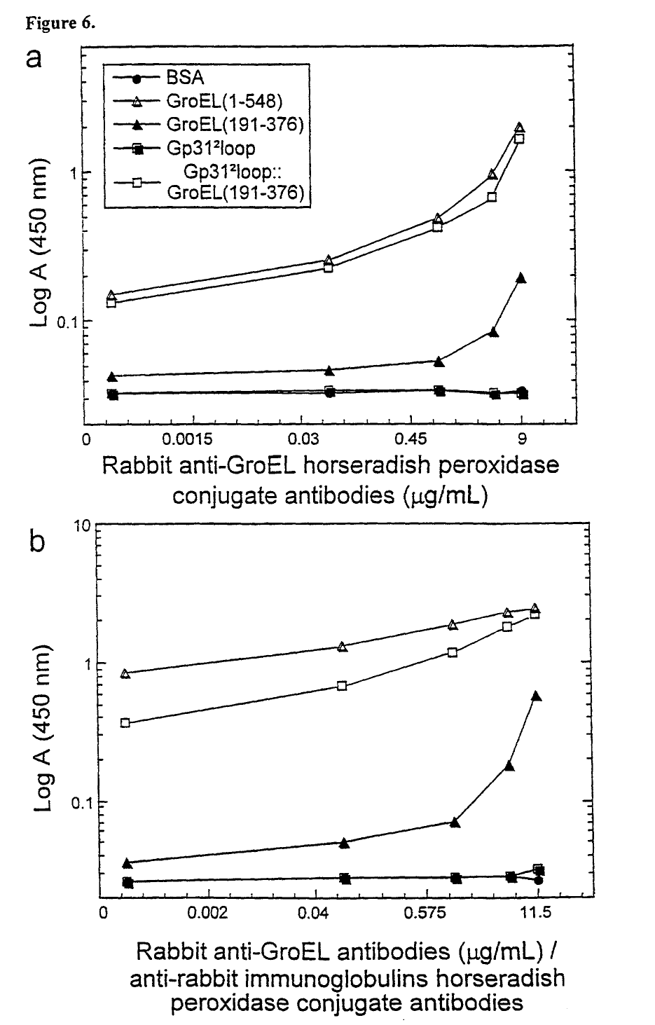

The functionality of MC₇ was examined for binding to GroES, since the interaction between GroEL and GroES is known to be less favourable for one monomer than for the heptamer. MC₇ bound specifically to GroES, conversely monomeric minichaperone GroEL(191-376) did not detectably bind the bacterial co-chaperonin (FIG. 5a).

The ability of a synthetic peptide corresponding to residues 16 to 32 of GroES mobile loop to displace bound GroES from MC₇ was tested by competition ELISA. The synthetic GroES mobile loop peptide did inhibit the binding of MC₇ with an IC₅₀ of 10 μM compared to 100 μM for GroEL (FIG. 5b). The apparent dissociation constant for the formation of the GroEL-GroES complex is low (10⁻⁶ M), which is compatible with cycling of GroES on and off GroEL during chaperonin-assisted folding. On the other hand, GroEL_{SR1} (Weissman, J. S., Rye, H. S., Fenton, W. A., Beechem, J. M. & Horwich, A. L. (1996) Cell 84, 481-490) is unable to release GroES in the absence of signal transmitted via the binding of ATP to an adjacent ring. The 10-fold decrease of the affinity of MC₇ for GroES may be sufficient for multiple binding and release cycles.

In vitro Activity of MC₇.

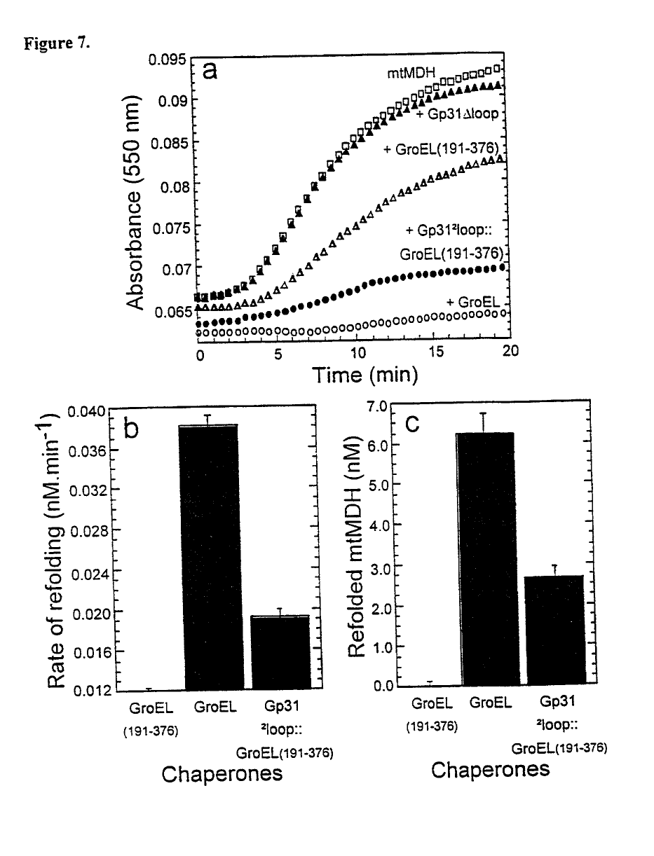

In vitro, heat- and dithiothreitol-denatured mitochondrial malate dehydrogenase (mtMDH) refolds in high yield only in the presence of GroEL, ATP, and the co-chaperonin GroES (Peres Ben-Zvi, A. P., Chatellier, J., Fersht, A. R. & Goloubinoff, P. (1998) Proc. Natl. Acad. Sci. U.S.A. 95, 15275-15280). Monomeric minichaperone GroEL(191-376) binds denatured mtMDH, protecting is aggregation (FIG. 7a) but, it is ineffective in enhancing the refolding rate (FIG. 7b). Conversely, MC₇, which protect further denatured mtMDH from aggregation (FIG. 7a) is active in refolding denatured mtMDH (FIG. 7a) with a rate of 0.02 nM.min⁻¹, compared to 0.04 nM.min⁻¹ for wild-type GroEL alone (FIG. 7b). After 120 min, the yield of refolded mtMDH by MC₇ is about 2.5-3 nM, compared to 6 nM of enzyme rescued by wild-type GroEL (FIG. 7c). Although saturating concentration of GroES (4 μM) does increase about 3- to 5-fold the rates at the beginning of the refolding reaction, a 10-fold decrease of the final yield was observed; indicating the absence of multiple cycles of binding and release of GroES to MC₇ (data not shown). Nevertheless, MC₇ is more efficient than GroEL_{SR1} mutant (Llorca, O., Perez-Perez, J., Carrascosa, J., Galan, A., Muga, A. & Valpuesta, J. (1997) J. Biol. Chem. 272, 32925-32932; Nielsen, K. L. & Cowan, N. J. (1998) Molecular Cell 2, 1-7; this study); remarkably, MC₇ is only 2-fold less active than wild-type GroEL in refolding a non-permissive substrate in vitro.

### Example 3

### In vivo Complementation of Thermosensitive groEL Mutant Alleles at 43° C.

We sought complementation of two thermosensitive (ts) groEL mutants of E. coli at 43° C. E. coli SV2 has the mutation Glu191→Gly in GroEL corresponding to groEL44 allele, while SV6 carries the EL673 allele, which has two mutations, Gly173→Asp and Gly337→Asp. Complementation experiments were performed by transforming the thermosensitive (ts) E. coli strains SV2 or SV6 with the pJC series of expression vectors vector (Chatellier, J., Hill, F., Lund, P. & Fersht, A. R. (1998) Proc. Natl. Acad. Sci. U.S.A. 95, 9861-9866) and plating an aliquot of the transformation reaction directly at 43° C. Subsequently, plasmids pJC from a representative number of individual clones growing at 43° C. were lost in the absence of continued chloramphenicol selection. Nearly all (≧95%) the cured clones were thermosensitive at 43° C. indicating the absence of recombination events for the reconstitution of wild-type groEL gene. The results obtained are qualitatively similar to those previously described (Chatellier, J., Hill, F., Lund, P. & Fersht, A. R. (1998) Proc. Natl. Acad. Sci. U.S.A. 95, 9861-9866). Only minichaperone sht-GroEL(193-335) complements the defect in SV2. The defective groEL in SV6 was complemented by expression of minichaperone sht-GroEL(191-345), and less well by sht-GroEL(193-335). Conversely, MC₇ and GroEL_{SR1} complement both temperature-sensitive E. coli groEL44 and groEL673 alleles at 43° C. (Table 1 ? Colony-forming units were not observed for either strain at 43° C. with vectors either lacking inserts (pJCsht) or lacking GroEL(1191-376) (pJCGp31Δloop).

It has been suggested the higher stability of shortest minichaperone sht-GroEL(193-335) could be responsible for the complementation of groEL44 mutant allele. To test this eventuality, we purified GroEL(E91 G; groEL44 allele) mutant and compared its thermal stability with the wild-type GroEL. We found no difference in stability between the mutant and the wild-type proteins in presence or absence of ATP. In addition, highly stable functional mutants of GroEL (193-345) do not complement, as the parental minichaperone (Table 1), the defects in SV2 or even SV6. We concluded the thermal stability of minichaperone is not accountable for the complementation of groEL defects.

TABLE 1
Relative colony forming ability of transformed ts groEL44 or groEL673
E. coli strains at 43° C.

| | GroEL strains | |
|---|---|---|
| Plasmids pJC | SV2 groEL44 | SV6 groEL673 |
| short his tag (sht) (ES⁻, EL⁻) | <10⁻⁴ | <10⁻⁴ |
| GroES(1-97) | 5 × 10⁻³ | <10⁻⁴ |
| Gp31(1-111) | 0.5 × 10⁻³ | <10⁻⁴ |
| Gp31 Δloop | <10⁻⁴ | <10⁻⁴ |
| GroEL(1-548) | 1 | 1 |
| GroES-EL | 1 | 1 |
| sht-GroEL(191-376) | <10⁻⁴ | <10⁻⁴ |
| sht-GroEL(191-345) | 0.01-0.02 | 0.07-0.09 |
| sht-GroEL(193-335) | 0.05-0.09 | 0.03-0.05 |
| Gp31Δ::GroEL(191-376) | 0.15-0.2 | 0.1 |

### Example 4

### In vivo Complementation at 37° C.

The effects of MC₇ on the growth at 37° C. of a strain of E. coli in which the chromosomal groEL gene had been deleted were analysed in two ways. First, we attempted to delete the groEL gene of TG1 which had been transformed with the different pJC MC₇ vector by PI transduction. However, no transductants could be obtained where the groEL gene had been deleted, unless intact GroEL was expressed from the complementing plasmid. This is consistent with the known essential role of GroEL. Second, we analysed the complementation of A190 (ΔgroEL::kan^{R}) [pBAD-EL] E. coli strain. In this strain, the chromosomal groEL gene has been deleted and GroEL is expressed exclusively from a plasmid-borne copy of the gene which can be tightly regulated by the arabinose P_{BAD} promoter and its regulatory gene, araC. AraC protein acts as either a repressor or an activator depending on the carbon source used. P_{BAD} is activated by arabinose but repressed by glucose (Guzman, L. -M., Belin, D., Carson, M. J. & Beckwith, J. (1995) J. Bacteriol. 177, 41214130). The A190 [pBAD-EL] cells can not grow on medium supplemented with glucose at 37° C. (Ivic, A., Olden, D., Wallington, E. J. & Lund, P. A. (1997) Gene 194, 1-8). As minichaperones (Chatellier, J., Hill, F., Lund, P. & Fersht, A. R. (1998) Proc. Natl. Acad. Sci. U.S.A. 95, 9861-9866.), MC₇ was unable to suppress this groEL growth defect (Table 2). We then determined whether MC₇ could supplement low levels of GroEL from transfected A190 [pBAD-EL]. At 0.01% arabinose, cells transfected with pJC expressing sht alone, Gp31Δloop or sht-GroEL(191-376), showed little colony forming ability (lees than 5%). But those containing pJC MC₇ produced about 30% of the number produced in the presence of 0.2% arabinose. Thus, pJC MC₇, but not pJCGroEL_{SR1}, significantly supplements depleted levels of GroEL., about twice as pJC sht-GroEL(193-335) (Chatellier, J., Hill, F., Lund, P. & Fersht, A. R. (1998) Proc. Natl. Acad. Sci. U.S.A. 95, 9861-9866).

TABLE 2
Plating ability of transformed AI90 (ΔgroEL::kan^{R}) [pBADEL]
E. coli strain at 37° C. in presence of different amount of arabinose.

| | % L(+)arabinose | | | |
|---|---|---|---|---|
| Plasmids pJC | 0.15 | 0.10 | 0.01 | 0.00 |
| short his tag (sht) | ++ | + | +/− | − |
| (ES⁻, EL⁻) | | | | |
| Gp31Δloop | ++ | + | +/− | − |
| GroEL (1-548) | +++ | +++ | +++ | +++ |
| sht-GroEL (191-376) | ++ | + | +/− | − |
| Gp31Δ:: | +++ | +++ | + | − |
| GroEL (191-376) | | | | |

+++, growth identical to that in presence of 0.2% L(+)arabinose (100%), in terms of both number and size; ++, about 50% of the colonies relative to that in presence of 0.2% L(+)arabinose; +, about 30% of the colonies; +/−, ≦5% of the colonies and size reduced relative to that in presence of 0.2% L(+)arabinose; −, no visible colonies.

### Example 5

### Effect on Bacteriophages λ and T4 growth of over-expressing MC₇.

Bacteriophages λ and T4 require the chaperonins GroES and GroEL for protein folding during morphogenesis (Zeilstra-Ryalls, J., Fayet, O. & Georgopoulos, C. (1991) Annu. Rev. Microbiol. 45, 301-325). Nine groE alleles which fail to support λ growth have been sequenced (Zeilstra-Ryalls, J., Fayet, O., Baird, L. & Georgopoulos, C. (1993) J. Bacteriol. 175, 1134-1143). We examined the ability of MC₇, over-expressed from the constitutive tet promoter on a high-copy number vector (see FIG. 2), to complement three mutant groEL alleles for plaque formation by λ (b₂cI) at 30° C. (Table 3) and T4 at 37° C. (Table 4).

The groE operon was named for its effects on the E protein of λ (Georgopoulos, C., Hendrix, R. W., Casjens, S. R. & Kaiser, A. D. (1973) J. Mol. Biol. 76, 45-60). Although heat induction of the groE operon has been shown to decrease burst size of λ bacteriophage in E. coli (Wegrzyn, A., Wegrzyn, G. & Taylor, K. (1996) Virology 217, 594-597). In contrast, we showed that the over-expression of GroEL alone, which resulted in slower growth of the bacteria, suffices to inhibit λ growth (Table 3). This effect was specific; over-expression of GroEL together with GroES caused only a four-fold drop in plaques. Over-expression of GroES alone had no effect. Minichaperone GroEL(191-376) had no effect on plaque counts in SV1 (groE⁺). Conversely, over-expression of MC₇ prevents plaque formation by bacteriophage λ in SV1, but less markedly than GroEL (Table 3). It seems that the main effect of GroEL over-expression is mediated through the λ origin, which requires two proteins, O and P. As with GroEL, MC₇ (or GroEL_{SR1}) inhibit the replication of the Lorist6 plasmid which use the bacteriophage λ origin. The effect on Lorist6 shows that the unfoldase activity is also an essential part of GroEL activity in vivo. MC₇ and minichaperones possess both, un- and folding, activities. GroEL over-expression gives weak complementation of λ growth in SV2 (groEL44) and SV3 (groEL59; Ser201→Phe). MC₇ does not, but GroEL_{SR1} does complement any of the E. Coli groEL mutant strains for bacteriophage λ growth at 30° C. (Table 3).

Bacteriophage T4 (T4D0) also requires a functional groEL gene, but encodes a protein Gp31 which can substitute for GroES. The requirement for GroEL can be distinguished genetically from λ's requirement. Thus only two of the four groEL alleles fail to support T4 replication; these are also the two thermosensitive mutations EL44 and EL673 (Zeilstra-Ryalls, J., Fayet, O., Baird, L. & Georgopoulos, C. (1993) J. Bacteriol. 175, 1134-1143; Zeilstra-Ryalls, J., Fayet, 0. & Georgopoulos, C. (1991) Annu. Rev. Microbiol. 45, 301-325). While over-expression of Gp31 allows T4 growth in all strains (only SV2 and SV6 strains normally do not allow T4 growth), over-expression of Gp31Δloop inhibits T4 replication. On the other hand, MC₇ does, as does GroEL_{SR1}, complement E. coli groEL mutant strains for bacteriophage T4 growth at 30° C. (Table 3).

An Unusual Basis for Thermo-Sensitivity in a groEL Mutation.

Surprisingly, over-expression of GroES demonstrates allele-specific complementation for λ and T4 of GroEL44 (Glu191→Gly) mutant (Tables 3 & 4). The effect is nevertheless incomplete; plaques on SV2 [pJCGroES] are invariably smaller than on SV1, or SV1 [pJCGroES]. The E191G single mutation blocks the assembly of the head structure of bacteriophage λ. A possible molecular basis for this allele-specificity lies in the nature of the groEL44 mutation. The substitution of Glu191→Gly in the hinge region between the intermediate and apical domains of GroEL presumably increases the flexibility of the hinge, and thereby, modulates a hinged conformational change in GroEL required for proper interaction with GroES. Indeed, the pivoting of the hinge region ensures proper interaction with GroES. For example, the mutant GroEL59 (Ser201→Phe in the same hinge region) in SV3 has low affinity for GroES. Over-expression of GroES will favour the formation of GroES-EL44 complex; we indeed also observed complementation of SV2 for thermosensitivity and bacteriophages growth by over-expressing GroEL44 mutant. Taking advantage of the GroES effect, we observed that GroEL minichaperones and MC₇ all reduce both plaque size and number but, like GroEL, do not completely eliminate them in SV2 [pBADGroES].

GroEL44, purified to homogeneity, is effective in refolding heat- and DTT-denatured mitochondrial malate dehydrogenase in presence of ATP and saturating concentration of GroES. Surprisingly, GroEL44 is as thermo-stable as the wild-type GroEL, indicating the mutation does not destabilise the overall conformation of the mutant. As anticipated from our in vivo genetic analysis, the affinity between GroEL44 and GroES is decreased at 37° C. and even more at higher temperature.

Our results suggest that the groEL44 mutation changes the distribution of GroEL subunits between apical domain-open and closed conformations. To allow GroEL_{SR1} to release GroES in the absence of signal transmitted via the binding of ATP to an adjacent ring, we introduced the Glu191→Gly mutation in GroEL_{SR1}, generating the GroEL_{SR2} mutant. GroEL_{SR2} is more efficient than MC₇ and even more than GroEL_{SR1} in vitro and in vivo.

TABLE 3
Growth of bacteriophage λ at 30° C. in transformed wild-type and
groEL mutant strains.

| | groEL strains | | | |
|---|---|---|---|---|
| | SV1 | SV2 | SV3 | SV6 |
| Plasmids pJC | (groEL⁺) | groEL44 | GroEL59 | groEL673 |
| short his tag (sht) | +++ | − | − | − |
| (ES⁻, EL⁻) | | | | |
| GroES (1-97) | +++ | +++ | − | − |
| Gp31 (1-111) | +++ | − | − | − |
| Gp31Δloop | +++ | − | − | − |
| GroEL (1-548) | − | + | ++ | +/− |
| sht-GroEL (191-376) | +++ | − | − | − |
| Gp31Δ::GroEL (191- | + | − | − | − |
| 376) | | | | |

TABLE 4
Growth of bacteriophage T4 at 37° C. in transformed wild-type and
groEL mutant strains.

| | groEL strains | | | |
|---|---|---|---|---|
| | SV1 | SV2 | SV3 | SV6 |
| Plasmids pJC | (groEL⁺) | groEL44 | GroEL59 | groEL673 |
| short his tag (sht) | +++ | − | +++ | − |
| (ES⁻, EL⁻) | | | | |
| GroES (1-97) | +++ | +++ | +++ | − |
| Gp31 (1-111) | +++ | +++ | +++ | +++ |
| Gp31Δloop | +/− | − | − | − |
| GroEL (1-548) | +++ | +++ | +++ | +++ |
| sht-GroEL (191-376) | +++ | − | +++ | − |
| Gp31Δ::GroEL (191- | ++ | + | + | +/− |
| 376) | | | | |

+++, normal plaque-forming ability relative to wild-type groEL⁺ strain, in terms of both number and size; ++, 5-fold fewer plaques relative to wild-type groEL⁺ strain, or both; +, 10-fold fewer plaques, or plaque size reduced relative to wild-type groEL⁺ strain, or both; +/−, 10²-fold fewer plaques and plaque size reduced relative to wild-type groEL⁺ strain; −, no visible plaques (<10⁻⁴).

### Example 6

### MC₇₂

A second oligomeric minichaperone polypeptide was constructed based on the GroES scaffold. This polypeptide, named MC₇₂, is GroESΔloop::GroEL(191-376).

Plasmid Constructions

Standard molecular biology procedures were used (Sambrook et al., 1989). The plasmid pRSETA encoding GroES gene has been described (Chatellier et al. 1998 In vivo activities of GroEL minichaperones. Proc. Natl. Acad. Sci. USA 95, 9861-9866). The GroES mutant Gly24Trp was generated by polymerase chain reaction (PCR), as described (Hemsley et al., 1989 A simple method for site-directed mutagenesis using the polymerase chain reaction. Nucl. Acids Res. 17, 6545-6551) using the template pRSETA encoding GroES (Chatellier et al., 1998) and the oligonucleotides 5′-C GGC TGG ATC GTT CTG ACC G-3′and 5′-GC AGA TTT AGT TTC AAC TTC TTT ACG-3′, creating a Nae I site (bold characters).

The DNA sequence encoding a part of the mobile loop of GroES (residues 16 to 33) was removed by PCR, as described (Hemsley et al., 1989), using the oligonucleotides 5′-TCC GGC TCT GCA GCG G-3′ and 5′-TCC AGA GCC AGT TTC AAC TTC TTT ACG C-3′, creating a unique BamHI site (bold characters) and the vector pRSET A-GroESΔloop.

The GroEL minichaperone gene (corresponding to the apical domain of GroEL, residues 191 to 376; Zahn et al., 1996 Chaperone activity and structure of monomeric polypeptide binding domains of GroEL Proc. Nat. Acad. Sci. USA 93, 15024-15029) was amplified by PCR and cloned into the unique BamHI site of pRSETA-GroESΔloop vector, thus inserting the minichaperone GroEL(191-376) in-frame into the GroESΔloop sequence. These genes were subcloned into the unique Nde I and Eag I sites of pACYC184, pJC and pBAD30 vectors (Guzman et al., 1995, Tight regulation, modulation, and high level expression by vectors containing the pBAD promoter. J. Bacteriol. 177, 4121-4130; Chatellier et al., 1998). PCR cycle sequencing using fluorescent dideoxy chain terminators (Applied Biosystems) was performed and analysed on an Applied Biosystems 373A machine. All PCR amplified DNA fragments were sequenced after cloning.

Proteins Expression, Purification and Characterization.

The GroES proteins, wild-type (˜10.4 kDa) and mutant Gly24Trp (˜10.5 kDa), Δloop (˜9.8 kDa), MC₇₂ (˜30 kDa), were expressed by inducing the T7 promoter of pRSETA-Eag I based vectors with isopropyl-β-D-thiogalactoside (IPTG) in E. coli C41(DE3) (Miroux & Walker, 1996 Over-production of proteins in Escherichia coli: Mutant hosts that allow synthesis of some membrane proteins and globular proteins at high levels. J. Mol. Biol. 260, 289-298) overnight at 25° C. and purified as described (Chatellier et al., 1998).

Proteins were analysed by electrospray mass spectrometry. Protein concentration was determined by absorbance at 276 nm using the method of Gill & von Hippel (1989 Calculation of protein extinction coefficients from amino acid sequence data. Anal. Biochem. 182, 319-326) and confirmed by quantitative amino acid analysis. In this study, protein concentrations refer to protomers, and not to oligomers.

Characterisation of MC₇₂

By both analytical size exclusion chromatography and analytical ultracentrifugation, both purified proteins, GroESΔloop and MC₇₂, were heptamers of seven 9.8 and seven 30 kDa subunits, respectively. The introduction into the GroES scaffold of a foreign polypeptide substantially larger than itself did not prevent oligomerisation. Electron microscopic studies of MC₇₂ revealed a diameter close to that of GroEL.

GroES Binding

The functionality of MC₇₂ was verified by examining GroES binding (followed by fluorescence) and mtMDH refolding.

### Example 7

### Reduction of Protein Aggregation in Huntingdon's Disease

Huntington's disease (HD), spinocerebellar ataxias types 1 and 3 (SCA1, SCA3), and spinobulbar muscular atrophy (SBMA) are caused by CAG/polyglutamine expansion mutations (Perutz, M. F. 1999 Trend Biochem. Sci. 24, 58-63; Rubinsztein, D. C. et al. 1999 J. Med. Genet. 36, 265-270). A feature of these diseases is ubiquitinated intraneuronal inclusions derived from the mutant proteins, which colocalize with heat shock proteins (HSPs) in SCA1 and SBMA and proteasomal components in SCA1, SCA3, and SBMA. Previous studies suggested that HSPs might protect against inclusion formation, because overexpression of HDJ-2/HSDJ (a human HSP40 homologue) reduced ataxin-1 (SCA1) and androgen receptor (SBMA) aggregate formation in HeLa cells (See Wyttenbach, A. et al. (2000) Proc. Natl. Acad. Sci. USA 97, 2899-2903).

These phenomena have been studied by transiently transfecting part of Huntingdon exon 1 in COS-7, PC12, and SH-SY5Y cells. Inclusion formation was not seen with constructs expressing 23 glutamines but was repeat length and time dependent for mutant constructs with 43-74 repeats. HSP70, HSP40, the 20S proteasome and ubiquitin colocalized with inclusions. Treatment with heat shock or with lactacystin, a proteasome inhibitor, increased the proportion of cells with inclusions of mutant huntingtin exon 1. Thus, inclusion formation may be enhanced in polyglutamine diseases, if the pathological process results in proteasome inhibition or a heat-shock response. Overexpression of HDJ-2/HSDJ did not modify inclusion formation in PC12 and SH-SY5Y cells but increased inclusion formation in COS-7 cells. To our knowledge, this is the first report of an HSP increasing aggregation of an abnormally folded protein in mammalian cells and expands the current understanding of the roles of HDJ-2yHSDJ in protein folding (See Wyttenbach, A. et al. (2000) Proc. Natl. Acad. Sci. USA 97, 2899-2903).

In the eucaryotic cell, molecular chaperones might be involved in the actual formation of nuclear aggregates by stabilizing the unfolded protein in an intermediate conformation which has the propensity to interact with neighbouring, unfolded proteins (Chirmer, E. C. & Lindquist, S. 1997 Proc. Natl. Acad. Sci. USA 94: 13932-7; DebBurman, S. K. et al., 1997 Proc. Natl. Acad. Sci. USA 94: 1393843; Welch, W. J. & Gambetti, P. 1998 Nature 392: 234). The chaperone's dual roles in aggregate formation and suppression may not be mutually exclusive, but rather dependent on the presence and level of chaperone expression. For example, the yeast chaperone Hsp104 (or bacterial GroEL) was shown to be necessary, at intermediate levels, for the propagation of the prion-like factor [PSI⁺], but when Hsp104 was overexpressed, [PSI⁺] was lost. Overexpression of the yeast homologue Hsp70 also inhibited [PSI⁺] (Chernoff, Y. O. et al., 1995 Science 268: 880-4). A similar phenomenon may occur in spinocerebellar ataxia type 1, with endogenous levels of HDJ2/HDJ and/or Hsc70 contributing to the formation of ataxin-1 aggregates when the number of glutamine repeats is in the disease-causing range. As in yeast, it may be necessary to upregulate or modulate the level of molecular chaperones to reduce aggregate formation in affected neurons (Cummings, C. J. et al., 1998 Nat. Genet. 19: 148-54).

Recently, D. Rubinsztein et al. have shown that the overexpression of GroEL(191-345) minichaperone monomer reduced slightly but significantly the proportion of mutant Huntingdon exon 1-expressing PC12 and SH-SY5Y cells with inclusions and also reduced cell death. We have tested MC₇₂ in the same system.

The overexpression of MC₇₂ [i.e. the fusion protein GroESΔloop::GroEL(191-376)], like yeast Hsp104, reduced inclusion formation and cell death even further in the same cells. On the other hand, the overexpression of wild-type GroEL alone, whose activity is regulated by its co-chaperone GroES and ATP hydrolysis, had no effect.

The failure of Hsps to release their substrates in polyQ disease may be a common feature indicating the use of chaperones as therapeutic agents in these cases.

1. A polypeptide monomer capable of oligomerisation, said monomer comprising a polypeptide sequence which potentiates protein folding inserted into the sequence of a subunit of an oligomerisable protein scaffold.

2. A polypeptide monomer according to claim 1, wherein the oligomerisable protein scaffold subunit it selected from the group consisting of bacteriophage T4 Gp31, Escherichia coli GroES and homologues thereof of the cpn10 family.

3. A polypeptide monomer according to claim 1, wherein the polypeptide sequence is inserted into the sequence of the oligomerisable protein scaffold subunit such that both the N and C termini of the polypeptide monomer are formed by the sequence of the oligomerisable protein scaffold subunit.

4. A polypeptide monomer according to claim 1, wherein the polypeptide sequence is inserted into the oligomerisable protein scaffold subunit by replacing one or more amino acids thereof.

5. A polypeptide monomer according to claim 4, wherein the oligomerisable protein scaffold subunit is bacteriophage T4 Gp31 and the polypeptide sequence is inserted into the oligomerisable protein scaffold subunit by substantially replacing the mobile loop between amino acid positions 27 and 42.

6. A polypeptide monomer according to claim 4, wherein the oligomerisable protein scaffold subunit is Escherichia coli GroES and the polypeptide sequence is inserted into the oligomerisable protein scaffold subunit by substantially replacing the mobile loop between amino acid positions 19 and 29.

7. A polypeptide monomer according to claim 4, wherein the oligomerisable protein scaffold subunit is bacteriophage T4 Gp31 and the polypeptide sequence is inserted between positions 59 and 61 of the oligomerisable protein scaffold subunit.

8. A polypeptide monomer according to claim 4, wherein the oligomerisable protein scaffold subunit is Escherichia coli GroES and the polypeptide sequence is inserted between positions 56 and 57 of the oligomerisable protein scaffold subunit.

9. A polypeptide monomer according to claim 4, wherein the oligomerisable protein scaffold subunit is bacteriophage T4 Gp31 and polypeptide sequences are inserted between positions 27 and 42 and between 59 and 61.

10. A polypeptide monomer according to claim 4, wherein the oligomerisable protein scaffold subunit is Escherichia coli GroES and polypeptide sequences are inserted between positions 19 and 29 and 56 and 57.

11. A polypeptide monomer according to claim 2, wherein the polypeptide sequence is displayed at the N or C terminus of the oligomerisable protein scaffold subunit.

12. A polypeptide oligomer comprising two or more polypeptide monomers according to claim 1.

13. A polypeptide oligomer according to claim 12, which is a homooligomer.

14. A polypeptide oligomer according to claim 12, which is a heterooligomer.

15. A polypeptide oligomer according to claim 14, wherein complementary protein folding are juxtaposed through the oligomerisation of different polypeptide monomers.

16. A polypeptide oligomer according to claim 12, wherein the monomers are covalently crosslinked.

17. A polypeptide oligomer according to claim 12, wherein the protein scaffold is in the form of a ring.

18. A polypeptide oligomer according to claim 17, wherein the ring is a heptameric ring.

19. A polypeptide monomer or oligomer according to claim 1, wherein the polypeptide sequence is selected from the group consisting of a minichaperone, a protease prosequence and a foldase.

20. A polypeptide oligomer or monomer according to claim 19, wherein the foldase is selected from the group consisting of a thiol/disulphide oxidoreductase and a peptidyl prolyl isomerase.

21. A method for promoting the folding of a polypeptide comprising contacting the polypeptide with a polypeptide oligomer or monomer according to claim 19.

22. A method according to claim 21, wherein the polypeptide is an unfolded or misfolded polypeptide.

23. A method according to claim 21, wherein the polypeptide comprises a disulphide.

24. A method according to claim 21, wherein the foldase is selected from the group consisting of thiol/disulphide oxidoreductases and peptidyl-prolyl isomerases.

25. A method according to claim 24, wherein the thiol/disulphide oxidoreductase is selected from the group consisting of E. coli DsbA and mammalian PDI, or a derivative thereof.

26. A method according to claim 24, wherein the peptidyl prolyl isomerase is selected from the group consisting of cyclophilin, parbulen, SurA and FK506 binding proteins.

27. A method according to claim 21, comprising a polypeptide sequence with a polypeptide oligomer comprising a polypeptide monomer or two or more polypeptide monomers and a non-oligomerised foldase, said monomer or monomers comprising a polypeptide sequence which potentiates protein folding inserted into the sequence of a subunit of an oligomerisable protein scaffold.

28. A method according to claim 21, wherein the polypeptide oligomer, said oligomer comprising a polypeptide monomer or two or more polypeptide monomers, said monomer or monomers comprising a polypeptide sequence which potentiates protein folding inserted into the sequence of a subunit of an oligomerisable protein scaffold, and/or the foldase is immobilised onto a solid phase support.

29. A method according to claim 28 wherein the solid phase support is agarose.

30. A solid phase support having immobilised thereon a polypeptide oligomer according to claim 12 and/or a foldase.

31. A column packed at least in part with a solid phase support according to claim 30.

32. A method of promoting the folding of a polypeptide comprising utilizing a polypeptide according to claim 12, optionally in combination with a foldase.

33. The method according to claim 32 wherein the polypeptide and/or the foldase is immobilised on a solid phase support, wherein the polypeptide comprises a monomer or two or more polypeptide monomers, said monomer or monomers comprising a polypeptide sequence which potentiates protein folding inserted into the sequence of a subunit of an oligomerisable protein scaffold.

34. A composition comprising a combination of a polypeptide oligomer according to claim 12 and a foldase.
